SEQUENCE LISTING (10 sequences)

SEQ ID NO: 1 - protein, 9 aa, Artificial Sequence
  An artificially synthesized peptide sequence
VLPPRQPSA

SEQ ID NO: 2 - protein, 9 aa, Artificial Sequence
  An artificially synthesized peptide sequence
LMMSTLPKS

SEQ ID NO: 3 - protein, 9 aa, Artificial Sequence
  An artificially synthesized peptide sequence
RIICEAHKV

SEQ ID NO: 4 - protein, 10 aa, Artificial Sequence
  An artificially synthesized peptide sequence
KLMMSTLPKS

SEQ ID NO: 5 - DNA, 2731 bp, Homo sapiens
gcgcttggcgggagatagaaaagtgcttcaacccgcgccggcggcgactgcagttcctgcgagcgaggagcgcgggacctgctgacacgctgacgccttcgagcgcggcccggggcccggagcggccggagcagcccgggtcctgaccccggcccggctcccgctccgggctctgccggcgggcgggcgagcgcggcgcggtccgggccggggggatgtctcggcggacgcgctgcgaggatctggatgagctgcactaccaggacacagattcagatgtgccggagcagagggatagcaagtgcaaggtcaaatggacccatgaggaggacgagcagctgagggccctggtgaggcagtttggacagcaggactggaagttcctggccagccacttccctaaccgcactgaccagcaatgccagtacaggtggctgagagttttgaatccagaccttgtcaaggggccatggaccaaagaggaagaccaaaaagtcatcgagctggttaagaagtatggcacaaagcagtggacactgattgccaagcacctgaagggccggctggggaagcagtgccgtgaacgctggcacaaccacctcaaccctgaggtgaagaagtcttgctggaccgaggaggaggaccgcatcatctgcgaggcccacaaggtgctgggcaaccgctgggccgagatcgccaagatgttgccagggaggacagacaatgctgtgaagaatcactggaactctaccatcaaaaggaaggtggacacaggaggcttcttgagcgagtccaaagactgcaagcccccagtgtacttgctgctggagctcgaggacaaggacggcctccagagtgcccagcccacggaaggccagggaagtcttctgaccaactggccctccgtccctcctaccataaaggaggaggaaaacagtgaggaggaacttgcagcagccaccacatcgaaggaacaggagcccatcggtacagatctggacgcagtgcgaacaccagagcccttggaggaattcccgaagcgtgaggaccaggaaggctccccaccagaaacgagcctgccttacaagtgggtggtggaggcagctaacctcctcatccccgctgtgggttctagcctctctgaagccctggacttgatcgagtcggaccctgatgcttggtgtgacctgagtaaatttgacctccctgaggaaccatctgcagaggacagtatcaacaacagcctagtgcagctgcaagcgtcacatcagcagcaagtcctgccaccccgccagccttccgccctggtgcccagtgtgaccgagtaccgcctggatggccacaccatctcagacctgagccggagcagccggggcgagctgatccccatctcccccagcactgaagtcgggggctctggcattggcacaccgccctctgtgctcaagcggcagaggaagaggcgtgtggctctgtcccctgtcactgagaatagcaccagtctgtccttcctggattcctgtaacagcctcacgcccaagagcacacctgttaagaccctgcccttctcgccctcccagtttctgaacttctggaacaaacaggacacattggagctggagagcccctcgctgacatccaccccagtgtgcagccagaaggtggtggtcaccacaccactgcaccgggacaagacacccctgcaccagaaacatgctgcgtttgtaaccccagatcagaagtactccatggacaacactccccacacgccaaccccgttcaagaacgccctggagaagtacggacccctgaagcccctgccacagaccccgcacctggaggaggacttgaaggaggtgctgcgttctgaggctggcatcgaactcatcatcgaggacgacatcaggcccgagaagcagaagaggaagcctgggctgcggcggagccccatcaagaaagtccggaagtctctggctcttgacattgtggatgaggatgtgaagctgatgatgtccacactgcccaagtctctatccttgccgacaactgccccttcaaactcttccagcctcaccctgtcaggtatcaaagaagacaacagcttgctcaaccagggcttcttgcaggccaagcccgagaaggcagcagtggcccagaagccccgaagccacttcacgacacctgcccctatgtccagtgcctggaagacggtggcctgcggggggaccagggaccagcttttcatgcaggagaaagcccggcagctcctgggccgcctgaagcccagccacacatctcggaccctcatcttgtcctgaggtgttgagggtgtcacgagcccattctcatgtttacaggggttgtgggggcagagggggtctgtgaatctgagagtcattcaggtgacctcctgcagggagccttctgccaccagcccctccccagactctcaggtggaggcaacagggccatgtgctgccctgttgccgagcccagctgtgggcggctcctggtgctaacaacaaagttccacttccaggtctgcctggttccctccccaaggccacagggagctccgtcagcttctcccaagcccacgtcaggcctggcctcatctcagaccctgcttaggatgggggatgtggccaggggtgctcctgtgctcaccctctcttggtgcatttttttggaagaataaaattgcctctctcttaaaaaaaaaaaaaaaaaa

SEQ ID NO: 6 - protein, 700 aa, Homo sapiens
MSRRTRCEDLDELHYQDTDSDVPEQRDSKCKVKWTHEEDEQLRALVRQFGQQDWKFLASHFPNRTDQQCQYRWLRVLNPDLVKGPWTKEEDQKVIELVKKYGTKQWTLIAKHLKGRLGKQCRERWHNHLNPEVKKSCWTEEEDRIICEAHKVLGNRWAEIAKMLPGRTDNAVKNHWNSTIKRKVDTGGFLSESKDCKPPVYLLLELEDKDGLQSAQPTEGQGSLLTNWPSVPPTIKEEENSEEELAAATTSKEQEPIGTDLDAVRTPEPLEEFPKREDQEGSPPETSLPYKWVVEAANLLIPAVGSSLSEALDLIESDPDAWCDLSKFDLPEEPSAEDSINNSLVQLQASHQQQVLPPRQPSALVPSVTEYRLDGHTISDLSRSSRGELIPISPSTEVGGSGIGTPPSVLKRQRKRRVALSPVTENSTSLSFLDSCNSLTPKSTPVKTLPFSPSQFLNFWNKQDTLELESPSLTSTPVCSQKVVVTTPLHRDKTPLHQKHAAFVTPDQKYSMDNTPHTPTPFKNALEKYGPLKPLPQTPHLEEDLKEVLRSEAGIELIIEDDIRPEKQKRKPGLRRSPIKKVRKSLALDIVDEDVKLMMSTLPKSLSLPTTAPSNSSSLTLSGIKEDNSLLNQGFLQAKPEKAAVAQKPRSHFTTPAPMSSAWKTVACGGTRDQLFMQEKARQLLGRLKPSHTSRTLILS

SEQ ID NO: 7 - DNA, 22 bp, Artificial Sequence
  An artificially synthesized sequence
gtctaccaggcattcgcttcat

SEQ ID NO: 8 - DNA, 24 bp, Artificial
  An artificially synthesized sequence
tcagctggaccacagccgcagcgt

SEQ ID NO: 9 - DNA, 21 bp, Artificial
  An artificially synthesized sequence
tcagaaatcctttctcttgac

SEQ ID NO: 10 - DNA, 24 bp, Artificial
  An artificially synthesized sequence
ctagcctctggaatcctttctctt

(19) United States
(12) Patent Application Publication
(10) Pub. No.: US 2012/0328638 A1
(43) Pub. Date: Dec. 27, 2012

(54) MYBL2 PEPTIDES AND VACCINES CONTAINING THE SAME

(75) Inventors: Yusuke Nakamura, Tokyo (JP); Takuya Tsunoda, Kanagawa (JP); Ryuji Ohsawa, Kanagawa (JP); Sachiko Yoshimura, Kanagawa (JP); Tomohisa Watanabe, Kanagawa (JP)

(73) Assignee: Onco Therapy Science, Inc., Kawasaki-shi (JP)

(21) Appl. No.: 13/513,543

(22) PCT Filed: Dec. 2, 2010
(86) PCT No.: PCT/JP2010/007028

§ 371 Date: Aug. 17, 2012

Related U.S. Application Data
(60) Provisional application No. 61/266,871, filed on Dec. 4, 2009.

(51) Int. Cl. A61K 39/00; C12N 15/12; A61P 35/00; C12N 5/0783; C12N 5/0784; C12N 15/85; C12N 5/10; C07K 7/08; A61K 31/7088

(52) U.S. Cl. 424/185.1; 530/328; 536/23.5; 435/375; 435/455; 435/373; 435/325

(57) ABSTRACT

Peptide vaccines against cancer are described herein. In particular, epitope peptides derived from the MYBL2 gene that bind to HLA antigen and have cytotoxic T lymphocyte (CTL) inducibility, more particularly peptides having the amino acid sequence of SEQ ID NO: 5 and fragments thereof, are provided. The present invention further extends to peptides that include one, two, or several amino acid insertions, substitutions or additions to the aforementioned peptides or fragments, provided they retain cytotoxic T cell inducibility. Also provided as nucleic acids encoding any of the aforementioned peptides, antigen-presenting cells and isolated CTLs that target such peptides, and pharmaceutical agents and compositions including any of the aforementioned peptides, nucleic acids, and APCs as active ingredients. The components of the present invention have particular utility in connection with the treatment and/or prophylaxis (i.e., prevention) of cancers (tumors), and/or the prevention of a postoperative recurrence thereof.

### PRIORITY

The present application claims the benefit of U.S. Provisional Applications No. 61/266,871, filed on Dec. 4, 2009, the entire contents of which are incorporated by reference herein.

### TECHNICAL FIELD

The present invention relates to the field of biological science, more specifically to the field of cancer therapy. In particular, the present invention relates to novel peptides that are extremely effective as cancer vaccines, and drugs for treating and preventing tumors.

### BACKGROUND ART

It has been demonstrated that CD8 positive CTLs recognize epitope peptides derived from tumor-associated antigens (TAAs) on the major histocompatibility complex (MHC) class I molecule, and then kill the tumor cells. Since the discovery of melanoma antigen (MAGE) family as the first example of TAAs, many other TAAs have been discovered through immunological approaches (NPL 1, Boon T, Int J Cancer 1993 May 8, 54(2): 177-80; NPL 2, Boon T & van der Bruggen P, J Exp Med 1996 Mar. 1, 183(3): 725-9), and some of these TAAs are now in the process of clinical development as immunotherapeutic targets.

Identification of new TAAs capable of inducing potent and specific anti-tumor immune responses warrants further development and clinical investigation of peptide vaccination strategies for various types of cancer is ongoing (NPL 3, Harris C C, J Natl Cancer Inst 1996 Oct. 16, 88(20): 1442-55; NPL 4, Butterfield L H et al., Cancer Res 1999 Jul. 1, 59(13): 3134-42; NPL 5, Vissers J L et al., Cancer Res 1999 Nov. 1, 59(21): 5554-9; NPL 6, van der Burg S H et al., J Immunol 1996 May 1, 156(9): 3308-14; NPL 7, Tanaka F et al., Cancer Res 1997 Oct. 15, 57(20): 4465-8; NPL 8, Fujie T et al., Int J Cancer 1999 Jan. 18, 80(2): 169-72; NPL 9, Kikuchi M et al., Int J Cancer 1999 May 5, 81(3): 459-66; NPL 10, Oiso M et al., Int J Cancer 1999 May 5, 81(3): 387-94). To date, several clinical trials using these tumor-associated antigen (TAA) derived peptides have been reported. Unfortunately, many of the current cancer vaccine trials have shown only a low objective response rate (NPL 11, Belli F et al., J Clin Oncol 2002 Oct. 15, 20(20): 4169-80; NPL 12, Coulie P G et al., Immunol Rev 2002 October 188: 33-42; NPL 13, Rosenberg S A et al., Nat Med 2004 September, 10(9): 909-15). Accordingly, there remains a need for new TAAs as immunotherapeutic targets.

Through the screening of cDNA libraries with c-myb proto-oncogene probes, MYBL2 (GenBank Accession No: NM_{—}002466, SEQ ID NO: 5 encoding SEQ ID NO: 6), v-myb myeloblastosis viral oncogene homolog (avian)-like 2, has been identified as a member of the MYB family of transcriptional factor genes (NPL 14, Nomura N et al., Nucleic Acids Res. 1988 Dec. 9, 16(23): 11075-11089). MYBL2 is known to regulate cell cycle progression, and cyclin-driven phosphorylation by CDK2-cyclin A and CDK2-cyclin E complexes (NPL 15, Robinson C et al., Oncogene 1996 May 2; 12(9):1855-64, NPL 16, Lane et al., Oncogene 1997 May 22; 14(20):2445-53, NPL 17, Sala et al., Proc Natl Acad Sci 1997 Jan. 21; 94(2): 532-536, NPL 18, Johnson K et al., J Biol Chem 1999 Dec. 17; 274(51):36741-9). Recent reports have shown that Mip/LIN-9 regulates the expression of MYBL2 and that both proteins play a key role in the promotion of cell cycle progression through the control of S and M phase cyclins (NPL 19, Pilkinton M et al., J Biol Chem 2007 Jan. 5; 282(1):168-75). In addition, through analysis of gene expression profiles with a genome-wide cDNA microarray containing 23,040 genes, MYBL2 has been identified as a novel molecule up-regulated in several cancers, including, for example, testicular tumor, pancreatic cancer, bladder cancer, non-small cell lung cancer, small cell lung cancer and esophageal cancer (PTL 1, U.S. 60/937,616, PTL 2, WO 2004/031410). It is considered that MYBL2 is a novel oncoantigen and the epitope peptide derived from MYBL2 might be applicable to cancer immunotherapy for patients with several type tumors.

### CITATION LIST

### Patent Literature

[PTL 1] U.S. 60/937,616
[PTL 2] WO 2004/031410

### Non Patent Literature

[NPL 1] Boon T, Int J Cancer 1993 May 8, 54(2): 177-80
[NPL 2] Boon T & van der Bruggen P, J Exp Med 1996 Mar. 1, 183(3): 725-9
[NPL 3] Harris C C, J Natl Cancer Inst 1996 Oct. 16, 88(20): 1442-55
[NPL 4] Butterfield L H et al., Cancer Res 1999 Jul. 1, 59(13): 3134-42
[NPL 5] Vissers J L et al., Cancer Res 1999 Nov. 1, 59(21): 5554-9
[NPL 6] van der Burg S H et al., J Immunol 1996 May 1, 156(9): 3308-14
[NPL 7] Tanaka F et al., Cancer Res 1997 Oct. 15, 57(20): 4465-8
[NPL 8] Fujie T et al., Int J Cancer 1999 Jan. 18, 80(2): 169-72
[NPL 9] Kikuchi M et al., Int J Cancer 1999 May 5, 81(3): 459-66
[NPL 10] Oiso M et al., Int J Cancer 1999 May 5, 81(3): 387-94
[NPL 11] Belli F et al., J Clin Oncol 2002 Oct. 15, 20(20): 4169-80
[NPL 12] Coulie P G et al., Immunol Rev 2002 October, 188: 33-42
[NPL 13] Rosenberg S A et al., Nat Med 2004 September, 10(9): 909-15
[NPL 14] Nomura N et al., Nucleic Acids Res. 1988 Dec. 9, 16(23): 11075-11089
[NPL 15] Robinson C et al., Oncogene 1996 May 2; 12(9):1855-64
[NPL 16] Lane et al., Oncogene 1997 May 22; 14(20):2445-53
[NPL 17] Sala et al., Proc Natl Acad Sci 1997 Jan. 21; 94(2): 532-536
[NPL 18] Johnson K et al., J Biol Chem 1999 Dec. 17; 274(51):36741-9
[NPL 19] Pilkinton M et al., J Biol Chem 2007 Jan. 5; 282(1):168-75

### SUMMARY OF INVENTION

The present invention is based in part on the discovery of suitable targets of immunotherapy. Because TAAs are generally perceived for the immune system as “self” and therefore often have no immunogenicity, the discovery of appropriate targets is of extreme importance. As noted above, MYBL2 (SEQ ID NO: 6 encoded by the gene of GenBank Accession No. NM_{—}002466 (SEQ ID NO: 5)) has been identified as up-regulated in tissues of cancers, such as testicular tumor, pancreatic cancer, bladder cancer, non-small cell lung cancer, small cell lung cancer and esophageal cancer. Thus, MYBL2 is a candidate target of cancer/tumor immunotherapy.

The present invention is based, at least in part, on the identification of specific epitope peptides of MYBL2 that possess the ability to induce cytotoxic T lymphocytes (CTLs) specific to MYBL2. As discussed in detail below, peripheral blood mononuclear cells (PBMCs) obtained from a healthy donor were stimulated using HLA-A2 binding candidate peptides derived from MYBL2. CTL lines with specific cytotoxicity against HLA-A2 positive target cells pulsed with each of candidate peptides were then established. Thus, these results demonstrate that these peptides are HLA-A2 restricted epitope peptides that can induce potent and specific immune responses against cells expressing MYBL2. Thus, these results demonstrate that MYBL2 is strongly immunogenic and the epitopes thereof are effective targets for tumor immunotherapy.

Accordingly, it is an object of the present invention to provide isolated peptides binding to HLA antigen, which is MYBL2 (SEQ ID NO: 6) or fragments thereof. The present peptides are expected to have CTL inducibility and, thus, can be used to induce CTL ex vivo or can be administered to a subject for inducing immune responses against cancers, examples of which include, but are not limited to, testicular tumor, pancreatic cancer, bladder cancer, non-small cell lung cancer, small cell lung cancer and esophageal cancer. Preferably, the peptides are nonapeptides or decapeptides, and more preferably, a nonapeptide or decapeptide having an amino acid sequence selected from among SEQ ID NOs: 2-4, especially the amino acid sequence of SEQ ID NO: 3 shown herein to have particularly strong CTL inducibility.

The present invention contemplates modified peptides, having an amino acid sequence of SEQ ID NOs: 2-4, wherein one, two or more amino acid(s) is/are substituted or added, so long as the modified peptides retain the original CTL inducibility. Further, the present invention provides isolated polynucleotides encoding any of the peptides of the present invention. These polynucleotides can be used for inducing antigen-expressing cells (APCs) with CTL inducibility or can be administered to a subject for inducing immune responses against cancers as well as the present peptides.

When administered to a subject, the present peptides are presented on the surface of APCs so as to induce CTLs targeting the respective peptides. Therefore, one object of the present invention is to provide agents including any peptides or polynucleotides of the present invention for inducing CTL. Furthermore, agents, substances, or compositions including any of the peptides or polynucleotides can be used for the treatment and/or prophylaxis of cancer, and/or for prevention of postoperative recurrence thereof, such cancers including, but not limited to, testicular tumor, pancreatic cancer, bladder cancer, non-small cell lung cancer, small cell lung cancer and esophageal cancer. Thus, it is yet another object of the present invention to provide pharmaceutical agents for the treatment and/or prophylaxis of cancer and/or prevention of the postoperative recurrence thereof, such agents including any of the peptides or polynucleotides of the present invention. Instead of or in addition to the present peptides or polynucleotides, the present agents or pharmaceutical agents may include, as the active ingredients, APCs or exosomes that present any of the present peptides.

The peptides or polynucleotides of the present invention may be used to induce APCs that present on the surface a complex of an HLA antigen and a present peptide, for example, by contacting APCs derived from a subject with the present peptide or introducing a polynucleotide encoding the present peptide into APCs. Such APCs have high CTL inducibility against the target peptides and are useful for cancer immunotherapy. Accordingly, it is another object of the present invention to provide methods for inducing APCs with CTL inducibility as well as APCs obtained by the methods.

It is a further object of the present invention to provide a method for inducing CTL, methods that include the step of co-culturing CD8-positive cells with APCs or exosomes presenting a peptide of the present invention on its surface or the step of introducing a gene that includes a polynucleotide encoding a T cell receptor (TCR) subunit binding to the present peptide. CTLs obtainable by such methods also find use in the treatment and/or prevention of cancer, examples of which include, but are not limited to, testicular tumor, pancreatic cancer, bladder cancer, non-small cell lung cancer, small cell lung cancer and esophageal cancer. Therefore, it is another object of the present invention to provide CTLs obtainable by the present methods.

It is yet another object of the present invention to provide methods for inducing an immune response against cancer in a subject in need thereof, such methods including the step of administering agents or compositions containing MYBL2 or fragments thereof, polynucleotides encoding MYBL2 or the fragments thereof, or exosomes or APCs presenting MYBL2 or the fragments thereof.

The applicability of the present invention extends to any of a number of diseases related to or arising from MYBL2 overexpression including cancer, examples of which include, but are not limited to, testicular tumor, pancreatic cancer, bladder cancer, non-small cell lung cancer, small cell lung cancer and esophageal cancer.

More specifically, the present invention provides followings:

[1] An isolated oligopeptide comprising an amino acid sequence selected from the group consisting of SEQ ID NOs: 2 to 4, wherein said oligopeptide binds an HLA antigen and has cytotoxic T lymphocyte (CTL) inducibility,

[2] An isolated oligopeptide, wherein said oligopeptide binds an HLA antigen and has cytotoxic T lymphocyte (CTL) inducibility, wherein said oligopeptide consists of an amino acid sequence selected from among SEQ ID NOs: 2 to 4, wherein 1, 2, or several amino acids are inserted, substituted, deleted or added,

[3] The oligopeptide of [2], wherein said oligopeptide has one or both of the following characteristics:

(a) the second amino acid from the N-terminus is selected from among leucine and methionine; and

(b) the C-terminal amino acid is selected from among valine and leucine,

[4] The isolated oligopeptide of [1] to [3], wherein the HLA antigen is HLA-A2,

[5] The isolated oligopeptide of [1] to [4], wherein said oligopeptide is a nonapeptide or decapeptide,

[6] An isolated polynucleotide encoding an oligopeptide of any one of [1] to [5],

[7] An agent for inducing CTL, wherein the agent comprises one or more of the oligopeptide(s) set forth in any one of [1] to [5], or one or more of the polynucleotides set forth in [6],

[8] A pharmaceutical agent for the treatment and/or prophylaxis of cancer, and/or the prevention of a postoperative recurrence thereof, wherein the agent comprises one or more of the oligopeptide(s) set forth in any one of [1] to [5], or one or more of the polynucleotides set forth in [6],

[9] The pharmaceutical agent of [8], which is formulated for the administration to a subject whose HLA antigen is HLA-A2,

[10] The pharmaceutical agent of [8] or [9], which is formulated for treating cancer, [11] A method for inducing an antigen-presenting cell (APC) with CTL inducibility, wherein the method comprises one of the following steps:

(a) contacting an APC with an oligopeptide of any one of [1] to [5] in vitro, ex vivo or in vivo; or

(b) introducing a polynucleotide encoding an oligopeptide of any one of [1] to [5] into an APC,

[12] A method for inducing CTL by any of the methods comprising at least one of the following steps:

(a) co-culturing CD8-positive T cells with APCs, which presents on its surface a complex of an HLA antigen and an oligopeptide of any one of [1] to [5];

(b) co-culturing CD8-positive T cells with exosomes, which presents on its surface a complex of an HLA antigen and an oligopeptide of any one of [1] to [5]; and

(c) introducing a gene that comprises a polynucleotide encoding a T cell receptor (TCR) subunit polypeptide binding to an oligopeptide of any one of [1] to [5] into a T cell,

[13] An isolated APC that presents on its surface a complex of an HLA antigen and an oligopeptide of any one of [1] to [5],

[14] The APC of [13], which is induced by the method of [11],

[15] An isolated CTL that targets a peptide of any one of [1] to [5],

[16] The CTL of [15], which is induced by the method of [12], and

[17] A method of inducing immune response against cancer in a subject comprising the step of: administering to the subject an agent comprising an oligopeptide of any one of [1] to [5], an immunologically active fragment thereof, or a polynucleotide encoding the oligopeptide or immunologically active fragment.

It is to be understood that both the foregoing summary of the invention and the following detailed description are of exemplified embodiments, and not restrictive of the invention or other alternate embodiments of the invention. In addition to the above, other objects and features of the invention will become more fully apparent when the following detailed description is read in conjunction with the accompanying figures and examples. However, it is to be understood that both the foregoing summary of the invention and the following detailed description are of exemplified embodiments, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

Various aspects and applications of the present invention will become apparent to the skilled artisan upon consideration of the brief description of the figures and the detailed description of the present invention and its preferred embodiments which follows.

FIG. 1 is composed of a series of photographs, (a) and (b), depicting the results of IFN-gamma ELISPOT assays on CTLs that were induced with peptides derived from MYBL2. The CTLs in well number #8 stimulated with MYBL2-A02-9-144 (SEQ ID NO: 3) (a) showed potent IFN-gamma production compared with the control. The square on the well of these pictures indicates that the cells from corresponding well were expanded to establish the CTL line. In contrast, as typical case of negative data, no specific IFN-gamma production was detected from the CTL stimulated with MYBL2-A02-9-355 (SEQ ID NO: 1) (b). In the figure, “+” indicates the IFN-gamma production against target cells pulsed with the appropriate peptide, and “−” indicates the IFN-gamma production against target cells not pulsed with any peptides.

FIG. 2 is a line graph depicting the results of an IFN-gamma ELISA assay demonstrating the IFN-gamma production of CTL line stimulated with MYBL2-A02-9-144 (SEQ ID NO: 3). The results demonstrate that the CTL line established by stimulation with the peptide show potent IFN-gamma production as compared with the control. In the figure, “+” indicates the IFN-gamma production against target cells pulsed with the appropriate peptide, and “−” indicates the IFN-gamma production against target cells not pulsed with any peptides.

FIG. 3 is a line graph depicting the IFN-gamma production of the CTL clone established by limiting dilution from the CTL lines stimulated with MYBL2-A02-9-144 (SEQ ID NO: 3). The results demonstrate that the CTL clone established by stimulation with the peptide show potent IFN-gamma production as compared with the control. In the figure, “+” indicates the IFN-gamma production against target cells pulsed with the appropriate peptide and “−” indicates the IFN-gamma production against target cells not pulsed with any peptides.

FIG. 4 is a line graph depicting specific CTL activity against the target cells that express MYBL2 and HLA-A*0201. COS7 cells transfected with HLA-A*0201 or with the full length of MYBL2 gene were prepared as the controls. The CTL clone established with MYBL2-A02-9-144 (SEQ ID NO: 3) showed specific CTL activity against COS7 cells transfected with both MYBL2 and HLA-A*0201 (black diamond). On the other hand, no significant specific CTL activity was detected against target cells expressing either HLA-A*0201 (triangle) or MYBL2 (circle).

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

In case of conflict, the present specification, including definitions, will control. In addition, the materials, methods, and examples are illustrative only and not intended to be limiting.

### I. DEFINITIONS

The words “a”, “an”, and “the” as used herein mean “at least one” unless otherwise specifically indicated.

The terms “polypeptide”, “peptide” and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” as used herein refers to naturally occurring and synthetic amino acids, as well as amino acid analogs and amino acid mimetics that similarly function to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotides”, “nucleotides” and “nucleic acids” are used interchangeably herein and, unless otherwise specifically indicated are similarly to the amino acids referred to by their commonly accepted single-letter codes.

The terms “agent” and “composition” as used interchangeably herein to refer to a product including the specified ingredients in the specified amounts, as well as any product that results, directly or indirectly, from combination of the specified ingredients in the specified amounts. Such terms, when used in relation to “pharmaceutical agent” and “pharmaceutical composition”, are intended to encompass products including the active ingredient(s), and any inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, in the context of the present invention, the terms pharmaceutical agent” and “pharmaceutical composition” refer to any product made by admixing a molecule or compound of the present invention and a pharmaceutically or physiologically acceptable carrier. The phrase “pharmaceutically acceptable carrier” or “physiologically acceptable carrier”, as used herein, means a pharmaceutically or physiologically acceptable material, composition, substance or vehicle, including but not limited to, a liquid or solid filler, diluent, excipient, solvent or encapsulating material, involved in carrying or transporting the subject scaffolded polypharmacophores from one organ, or portion of the body, to another organ, or portion of the body.

The term “active ingredient” herein refers to a substance in an agent or composition that is biologically or physiologically active. Particularly, in a pharmaceutical agent or composition, “active ingredient” refers to a substance that shows an objective pharmacological effect. For example, in case of pharmaceutical agents or compositions for use in the treatment or prevention of cancer, active ingredients in the agents or compositions may lead to at least one biological or physiologically action on cancer cells and/or tissues directly or indirectly. Preferably, such action may include reducing or inhibiting cancer cell growth, damaging or killing cancer cells and/or tissues, and so on. Typically, indirect effect of active ingredients is inductions of CTLs recognizing or killing cancer cells. Before formulated, “active ingredient” is also referred to as “bulk”, “drug substance” or “technical product”.

Unless otherwise defined, the term “cancer” refers to the cancers overexpressing MYBL2 gene, examples of which include, but are not limited to testicular tumor, pancreatic cancer, bladder cancer, non-small cell lung cancer, small cell lung cancer and esophageal cancer.

Unless otherwise defined, the terms “cytotoxic T lymphocyte”, “cytotoxic T cell” and “CTL” are used interchangeably herein and unless otherwise specifically indicated, refer to a sub-group of T lymphocytes that are capable of recognizing non-self cells (e.g., tumor cells, virus-infected cells) and inducing the death of such cells.

Unless otherwise defined, the terms “HLA-A02” refers to the HLA-A2 type containing the subtypes such as HLA-A*-0201 or HLA-A*-0206.

To the extent that the materials and methods of the present invention find utility in the context of the “treatment” of cancer, a treatment is deemed “efficacious” if it leads to clinical benefit such as, reduction in expression of an MYBL2 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

Unless otherwise defined, the term “kit” as used herein, is used in reference to a combination of reagents and other materials. It is contemplated herein that the kit may include microarray, chip, marker, and so on. It is not intended that the term “kit” be limited to a particular combination of reagents and/or materials.

As used herein, in the context of a subject or patient, the phrase “HLA-A2 positive” refers to that the subject or patient homozygously or heterozygously possess HLA-A2 antigen gene, and HLA-A2 antigen is expressed in cells of the subject or patient as an HLA antigen.

To the extent that the materials and methods of the present invention find utility in the context of the “prevention” and “prophylaxis” of cancer, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

In the context of the present invention, the treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, and the reduction or inhibition of metastasis. Effective treatment and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease. Unless otherwise defined, all technical and scientific terms used herein have the same meaning as commonly understood by one of ordinary skill in the art to which this invention belongs.

### II. PEPTIDES

To demonstrate that peptides derived from MYBL2 function as an antigen recognized by CTLs, peptides derived from MYBL2 (SEQ ID NO: 6) were analyzed to determine whether they were antigen epitopes restricted by HLA-A2 which are commonly encountered HLA alleles (Date Y et al., Tissue Antigens 47: 93-101, 1996; Kondo A et al., J Immunol 155: 4307-12, 1995; Kubo R T et al., J Immunol 152: 3913-24, 1994). Candidates of HLA-A2 binding peptides derived from MYBL2 were identified based on their binding affinities to HLA-A2. The following peptides are considered to be candidate peptides:

MYBL2-A02-9-597 (SEQ ID NO: 2)

MYBL2-A02-9-144 (SEQ ID NO: 3) and

MYBL2-A02-10-596 (SEQ ID NO: 4).

After in vitro stimulation of T-cells by dendritic cells (DCs) loaded with these peptides, CTLs were successfully established using the following peptide:

MYBL2-A02-9-144 (SEQ ID NO: 3).

These established CTLs show potent specific CTL activity against target cells pulsed with respective peptides. These results herein demonstrate that MYBL2 is an antigen recognized by CTL and that the peptides are epitope peptides of MYBL2 restricted by HLA-A2.

Since the MYBL2 gene is overexpressed in cancer cells and tissues, including, but not limited to, those of testicular tumor, pancreatic cancer, bladder cancer, non-small cell lung cancer, small cell lung cancer and esophageal cancer, but is not expressed in most normal organs, it constitutes a good target for immunotherapy. Thus, the present invention provides nonapeptides (peptides composed of nine amino acid residues) and decapeptides (peptides composed of ten amino acid residues) corresponding to CTLrecognized epitopes of MYBL2. Preferred examples of nonapeptides and decapeptides of the present invention include those peptides having an amino acid sequence selected from among SEQ ID NOs: 2-4, especially 3.

Generally, software programs presently available on the Internet, such as those described in Parker K C et al., J Immunol 1994 Jan. 1, 152(1): 163-75, can be used to calculate the binding affinities between various peptides and HLA antigens in silico. Binding affinity with HLA antigens can be measured as described, for example, in the references to Parker K C et al., J Immunol 1994 Jan. 1, 152(1): 163-75; and Kuzushima K et al., Blood 2001, 98(6): 1872-81. Methods for determining binding affinity are described, for example, in the Journal of Immunological Methods, 1995, 185: 181-190 and Protein Science, 2000, 9: 1838-1846. Therefore, one can select fragments derived from MYBL2, which have high binding affinity with HLA antigens using such software programs. Thus, the present invention encompasses peptides composed of any fragments derived from MYBL2 that bind with HLA antigens identified using such known programs. The peptide of the present invention may be the peptide composed of the full length of MYBL2.

The peptides of the present invention, particularly the nonapeptides and decapeptides of the present invention, can be flanked with additional amino acid residues so long as the resulting peptide retains its CTL inducibility. The amino acid residues to be flanked to the present peptides may be composed of any kind of amino acids so long as they do not impair the CTL inducibility of the original peptide. Thus, the present invention encompasses peptides which include the peptides derived from MYBL2 and have binding affinity to HLA antigens. Such peptides are typically less than about 40 amino acids, often less than about 20 amino acids, usually less than about 15 amino acids.

In general, the modification of one, two or more amino acids in a peptide will not influence the function of the peptide, and in some cases will even enhance the desired function of the original protein. In fact, modified peptides (i.e., peptides composed of an amino acid sequence in which one, two or several amino acid residues have been modified (i.e., substituted, deleted, added or inserted) as compared to an original reference sequence) have been known to retain the biological activity of the original peptide (Mark et al., Proc Natl Acad Sci USA 1984, 81: 5662-6; Zoller and Smith, Nucleic Acids Res 1982, 10: 6487-500; Dalbadie-McFarland et al., Proc Natl Acad Sci USA 1982, 79: 6409-13). Thus, in one embodiment, the peptides of the present invention may have both CTL inducibility and an amino acid sequence selected from among SEQ ID NOs: 2-4, especially 3, wherein one, two or even more amino acids are added, inserted, deleted and/or substituted.

Those skill in the art recognize that individual additions or substitutions to an amino acid sequence which alters a single amino acid or a small percentage of amino acids tend to result in the conservation of the properties of the original amino acid side-chain. As such, they are often referred to as “conservative substitutions” or “conservative modifications”, wherein the alteration of a protein results in a modified protein having a function analogous to the original protein. Conservative substitution tables providing functionally similar amino acids are well known in the art. Examples of amino acid side chain characteristics that are desirable to conserve include, for example, hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:

### 1) Alanine (A), Glycine (G);

2) Aspartic acid (D), Glutamic acid (E);

### 3) Aspargine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins 1984).

Such conservatively modified peptides are also considered to be peptides of the present invention. However, peptides of the present invention are not restricted thereto and can include non-conservative modifications, so long as the resulting modified peptide retains the CTL inducibility of the original peptide. Furthermore, modified peptides should not exclude CTL inducible peptides of polymorphic variants, interspecies homologues, and alleles of MYBL2.

To retain the requisite CTL inducibility one can modify (insert, delete, add and/or substitute) a small number (for example, 1, 2 or several) or a small percentage of amino acids. Herein, the term “several” means 5 or fewer amino acids, for example, 4, 3 or fewer. The percentage of amino acids to be modified is preferably 20% or less, more preferably 15% or less, even more preferably 10% or less, or 1 to 5%.

Moreover, peptides of the present invention can be inserted, substituted or added with amino acid residues or amino acid residues may be deleted to achieve a higher binding affinity. When used in the context of immunotherapy, the present peptides should be presented on the surface of a cell or exosome, preferably as a complex with an HLA antigen. Therefore, it is preferable to select peptides that not only induce CTLs but also possess high binding affinity to the HLA antigen. To that end, the peptides can be modified by substitution, insertion, and/or addition of the amino acid residues to yield a modified peptide having improved binding affinity. In addition to peptides that are naturally displayed, since the regularity of the sequences of peptides displayed by binding to HLA antigens is already known (J Immunol 1994, 152: 3913; Immunogenetics 1995, 41: 178; J Immunol 1994, 155: 4307), modifications based on such regularity can be introduced into the immunogenic peptides of the invention.

Peptides possessing high HLA-A2 binding affinity tend to have a leucine or methionine residue as the second amino acid from the N-terminus and a valine or leucine residue as the amino acid at the C-terminus. Thus, peptides having the amino acid sequences of SEQ ID NOs: 2-4 wherein the second amino acid from the N-terminus of the amino acid sequence of said SEQ ID NOs is substituted with leucine or methionine, and/or wherein the C-terminus of the amino acid sequence of said SEQ ID NOs is substituted with valine or leucine are encompassed by the present invention. Substitutions can be introduced not only at the terminal amino acids but also at the position of potential T cell receptor (TCR) recognition of peptides. Several studies have demonstrated that a peptide with amino acid substitutions can be equal to or better than the original, for example CAP1, p53₍₂₆₄₋₂₇₂₎, Her-2/neu₍₃₆₉₋₃₇₇₎ or gp100₍₂₀₉₋₂₁₇₎ (Zaremba et al. Cancer Res. 57, 4570-4577, 1997, T. K. Hoffmann et al. J. Immunol. (2002) Feb. 1; 168(3):1338-47. S. O. Dionne et al. Cancer Immunol immunother. (2003) 52: 199-206 and S. O. Dionne et al. Cancer Immunology, Immunotherapy (2004) 53, 307-314).

The present invention also contemplates the addition of one, two or several amino acids to the N and/or C-terminus of the described peptides. Such modified peptides having high HLA antigen binding affinity and retained CTL inducibility are also included in the present invention.

However, when the peptide sequence is identical to a portion of the amino acid sequence of an endogenous or exogenous protein having a different function, side effects such as autoimmune disorders and/or allergic symptoms against specific substances may be induced. Therefore, it is preferable to first perform homology searches using available databases to avoid situations in which the sequence of the peptide matches the amino acid sequence of another protein. When it becomes clear from the homology searches that there exists not even a peptide with 1 or 2 amino acid differences as compared to the objective peptide, the objective peptide can be modified in order to increase its binding affinity with HLA antigens, and/or increase its CTL inducibility without any danger of such side effects.

Although peptides having high binding affinity to the HLA antigens as described above are expected to be highly effective, the candidate peptides, which are selected according to the presence of high binding affinity as an indicator, are further examined for the presence of CTL inducibility. Herein, the phrase “CTL inducibility” indicates the ability of the peptide to induce CTLs when presented on antigen-presenting cells (APCs). Further, “CTL inducibility” includes the ability of the peptide to induce CTL activation, CTL proliferation, promote CTL lysis of target cells, and to increase CTL IFN-gamma production.

Confirmation of CTL inducibility is accomplished by inducing APCs carrying human MHC antigens (for example, B-lymphocytes, macrophages, and dendritic cells (DCs)), or more specifically DCs derived from human peripheral blood mononuclear leukocytes, and after stimulation with the peptides, mixing with CD8-positive cells, and then measuring the IFN-gamma produced and released by CTL against the target cells. As the reaction system, transgenic animals that have been produced to express a human HLA antigen (for example, those described in BenMohamed L, Krishnan R, Longmate J, Auge C, Low L, Primus J, Diamond D J, Hum Immunol 2000 August, 61(8): 764-79, can be used. For example, the target cells can be radio-labeled with ⁵¹Cr and such, and cytotoxic activity can be calculated from radioactivity released from the target cells. Alternatively, CTL inducibility can be assessed by measuring IFN-gamma produced and released by CTL in the presence of APCs that carry immobilized peptides, and visualizing the IFN-gamma on the media using anti-IFN-gamma monoclonal antibodies.

As a result of examining the CTL inducibility of the peptides as described above, it was discovered that nonapeptides or decapeptides selected from among the amino acid sequences indicated by SEQ ID NOs: 2-4, especially SEQ ID NO: 3, shown herein to have particularly high CTL inducibility as well as high binding affinity to an HLA antigen. Thus, these peptides are exemplified as preferred embodiments of the present invention.

Furthermore, the result of homology analysis showed that those peptides do not have significant homology with peptides derived from any other known human gene products. This lowers the possibility of unknown or undesired immune responses arising when used for immunotherapy. Therefore, also from this aspect, these peptides find use for eliciting immunity in cancer patients against MYBL2. Thus, the peptides of the present invention, preferably, peptides having an amino acid sequence selected from among SEQ ID NOs: 2-4, especially SEQ ID NO: 3.

In addition to the modification of the present peptides discussed above, the described peptides can be further linked to other substances, so long as they retain the CTL inducibility of the original peptide and, more preferably, also retain the requisite HLA binding. Exemplary substances include: peptides, lipids, sugar and sugar chains, acetyl groups, natural and synthetic polymers, etc. The present peptides can additionally contain modifications such as glycosylation, side chain oxidation, and/or phosphorylation, so long as the modifications do not destroy the biological activity of the original peptide. Such modifications may confer additional functions (e.g., targeting function, and delivery function) and/or stabilize the peptides.

For example, to increase the in vivo stability of a polypeptide, it is known in the art to introduce D-amino acids, amino acid mimetics or unnatural amino acids; this concept can also be adopted to the present polypeptides. The stability of a polypeptide can be assayed in a number of ways. For instance, peptidases and various biological media, such as human plasma and serum, can be used to test stability (see, e.g., Verhoef et al., Eur J Drug Metab Pharmacokin 1986, 11: 291-302).

When the peptides of the present intention include a cystein residue, the peptides tend to form dimers via a disulfide bond between SH groups of the cyctein residues. Therefore, dimers of the peptide of the present invention are also included in the peptides of the present invention.

Moreover, as noted above, among the modified peptides that are substituted, deleted or added by one, two or several amino acid residues, those having same or higher activity as compared to original peptides can be screened for or selected. The present invention, therefore, also provides the method of screening for or selecting modified peptides having same or higher activity as compared to original. An illustrative method may include the steps of:

a: substituting, deleting or adding at least one amino acid residue of a peptide of the present invention,

b: determining the activity of the peptide, and

c: selecting the peptide having same or higher activity as compared to the original.

Herein, the activity to be assayed may include HLA binding activity, APC or CTL inducibility and cytotoxic activity.

Herein, the peptides of the present invention can also be described as “MYBL2 peptide(s)” or “MYBL2 polypeptide(s)”.

### III. PREPARATION OF MYBL2 PEPTIDES

The peptides of the invention can be prepared using well known techniques. For example, the peptides can be prepared synthetically, using recombinant DNA technology or chemical synthesis. The peptides of the invention can be synthesized individually or as longer polypeptides composed of two or more peptides. The peptides can then be isolated i.e., purified or isolated so as to be substantially free of other naturally occurring host cell proteins and fragments thereof, or any other chemical substances.

The peptides of the present invention may contain modifications, such as glycosylation, side chain oxidation, or phosphorylation provided such modifications do not destroy the biological activity of the original peptides. Other illustrative modifications include incorporation of D-amino acids or other amino acid mimetics that can be used, for example, to increase the serum half life of the peptides. A peptide of the present invention can be obtained through chemical synthesis based on the selected amino acid sequence. Examples of conventional peptide synthesis methods that can be adapted to the synthesis include, but are not limited to:
(i) Peptide Synthesis, Interscience, New York, 1966;
(ii) The Proteins, Vol. 2, Academic Press, New York, 1976;
(iii) Peptide Synthesis (in Japanese), Maruzen Co., 1975;
(iv) Basics and Experiment of Peptide Synthesis (in Japanese), Maruzen Co., 1985;
(v) Development of Pharmaceuticals (second volume) (in Japanese), Vol. 14 (peptide synthesis), Hirokawa, 1991;
(vi) WO99/67288; and
(vii) Barany G. & Merrifield R. B., Peptides Vol. 2, “Solid Phase Peptide Synthesis”, Academic Press, New York, 1980, 100-118.

Alternatively, the present peptides can be obtained adapting any known genetic engineering methods for producing peptides (e.g., Morrison J, J Bacteriology 1977, 132: 349-51; Clark-Curtiss & Curtiss, Methods in Enzymology (eds. Wu et al.) 1983, 101: 347-62). For example, first, a suitable vector harboring a polynucleotide encoding the objective peptide in an expressible form (e.g., downstream of a regulatory sequence corresponding to a promoter sequence) is prepared and transformed into a suitable host cell. The host cell is then cultured to produce the peptide of interest. The peptide can also be produced in vitro adapting an in vitro translation system.

### IV. POLYNUCLEOTIDES

The present invention also provides a polynucleotide which encodes any of the aforementioned peptides of the present invention. These include polynucleotides derived from the natural occurring MYBL2 gene (GenBank Accession No. NM_{—}002466 (SEQ ID NO: 5)) as well as those having a conservatively modified nucleotide sequence thereof. Herein, the phrase “conservatively modified nucleotide sequence” refers to sequences which encode identical or essentially identical amino acid sequences. Due to the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a peptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a peptide is implicitly described in each disclosed sequence. The polynucleotide of the present invention can be composed of DNA, RNA, and derivatives thereof. A DNA is suitably composed of bases such as A, T, C, and G, and T is replaced by U in an RNA.

The polynucleotide of the present invention can encode multiple peptides of the present invention with or without intervening amino acid sequences in between. For example, the intervening amino acid sequence can provide a cleavage site (e.g., enzyme recognition sequence) of the polynucleotide or the translated peptides. Furthermore, the polynucleotide can include any additional sequences to the coding sequence encoding the peptide of the present invention. For example, the polynucleotide can be a recombinant polynucleotide that includes regulatory sequences required for the expression of the peptide or can be an expression vector (plasmid) with marker genes and such. In general, such recombinant polynucleotides can be prepared by the manipulation of polynucleotides through conventional recombinant techniques using, for example, polymerases and endonucleases.

Both recombinant and chemical synthesis techniques can be used to produce the polynucleotides of the present invention. For example, a polynucleotide can be produced by insertion into an appropriate vector, which can be expressed when transfected into a competent cell. Alternatively, a polynucleotide can be amplified using PCR techniques or expression in suitable hosts (see, e.g., Sambrook et al., Molecular Cloning: A Laboratory Manual, Cold Spring Harbor Laboratory, New York, 1989). Alternatively, a polynucleotide can be synthesized using the solid phase techniques, as described in Beaucage S L & Iyer R P, Tetrahedron 1992, 48: 2223-311; Matthes et al., EMBO J. 1984, 3: 801-5.

### V. EXOSOMES

The present invention further provides intracellular vesicles called exosomes, which present complexes formed between the peptides of this invention and HLA antigens on their surface. Exosomes can be prepared, for example, using the methods detailed in Japanese Patent Application Kohyo Publications Nos. Hei 11-510507 and WO99/03499, and can be prepared using APCs obtained from patients who are subject to treatment and/or prevention. The exosomes of this invention can be inoculated as vaccines, in a fashion similar to the peptides of this invention.

The type of HLA antigens contained in the complexes must match that of the subject requiring treatment and/or prevention. For example, in the Japanese population, HLA-A2 (particularly, A*0201 and also A*0206) is prevalent and therefore would be appropriate for treatment of a Japanese patient. The use of the A2 type that is highly expressed among the Japanese and Caucasian is favorable for obtaining effective results. Typically, in the clinic, the type of HLA antigen of the patient requiring treatment is investigated in advance, which enables the appropriate selection of peptides having high levels of binding affinity to the particular antigen, or having CTL inducibility by antigen presentation. Furthermore, in order to obtain peptides having both high binding affinity and CTL inducibility, substitution, insertion and/or addition of 1, 2, or several amino acids can be performed based on the amino acid sequence of the naturally occurring MYBL2 partial peptide. When using the A2 type HLA antigen for the exosome of the present invention, the peptides having a sequence of any one of SEQ ID NOs: 2-4, especially 3, find use.

### VI. ANTIGEN-PRESENTING CELLS (APCS)

The present invention also provides isolated APCs that present complexes formed between HLA antigens and the peptides of this invention on its surface. The APCs can be derived from patients who are subject to treatment and/or prevention, and can be administered as vaccines by themselves or in combination with other drugs including the peptides of this invention, exosomes, or CTLs.

The APCs are not limited to a particular kind of cells and include dendritic cells (DCs), Langerhans cells, macrophages, B cells, and activated T cells, which are known to present proteinaceous antigens on their cell surface so as to be recognized by lymphocytes. Since DC is a representative APC having the strongest CTL inducing action among APCs, DCs find use as the APCs of the present invention.

For example, the APCs of the present invention can be obtained by inducing DCs from peripheral blood monocytes and then contacting (stimulating) them with the peptides of this invention in vitro, ex vivo or in vivo. When the peptides of this invention are administered to the subjects, APCs that present the peptides of this invention are induced in the body of the subject. Therefore, the APCs of this invention can be obtained by collecting the APCs from the subject after administering the peptides of this invention to the subject. Alternatively, the APCs of this invention can be obtained by contacting APCs collected from a subject with the peptide of this invention.

The APCs of the present invention can be administered alone or in combination with other drugs including the peptides, exosomes or CTLs of this invention to a subject for inducing immune response against cancer in the subject. For example, the ex vivo administration can include steps of:

a: collecting APCs from a first subject,

b: contacting the APCs of step a, with the peptide and

c: administering the APCs of step b to a second subject.

The first subject and the second subject can be the same individual, or may be different individuals. The APCs obtained by step b can be administered as a vaccine for treating and/or preventing cancer including testicular tumor, pancreatic cancer, bladder cancer, non-small cell lung cancer, small cell lung cancer and esophageal cancer.

The present invention also provides a method or process for manufacturing a pharmaceutical composition inducing APCs, wherein the method includes the step of admixing or formulating the peptide of the invention with a pharmaceutically acceptable carrier.

According to an aspect of the present invention, the APCs have a high level of CTL inducibility. In the term of “high level of CTL inducibility”, the high level is relative to the level of that by APC contacting with no peptide or peptides which cannot induce the CTL. Such APCs having a high level of CTL inducibility can be prepared by a method which includes the step of transferring a polynucleotide encoding the peptide of this invention to APCs in vitro as well as the method mentioned above. The introduced genes can be in the form of DNAs or RNAs. Examples of methods for introduction include, without particular limitations, various methods conventionally performed in this field, such as lipofection, electroporation, and calcium phosphate method. More specifically, it can be performed as described in Cancer Res 1996, 56: 5672-7; J Immunol 1998, 161: 5607-13; J Exp Med 1996, 184: 465-72; Published Japanese Translation of International Publication No. 2000-509281. By transferring the gene into APCs, the gene undergoes transcription, translation, and such in the cell, and then the obtained protein is processed, and proceeds through a presentation pathway to present peptides.

### VII. CYTOTOXIC T LYMPHOCYTES (CTLS)

A CTL induced against any of the peptides of the present invention strengthens the immune response targeting cancer cells in vivo and thus can be used as vaccines in a fashion similar to the peptides per se. Thus, the present invention also provides isolated CTLs that are specifically induced or activated by any of the present peptides. Such CTLs can be obtained by (1) administering the peptide(s) of the present invention to a subject, collecting CTLs from the subject; or (2) contacting (stimulating) subject-derived APCs, and CD8-positive cells, or peripheral blood mononuclear leukocytes in vitro with the peptide(s) of the present invention and then isolating CTLs; or (3) contacting CD8-positive cells or peripheral blood mononuclear leukocytes in vitro with APCs or exosomes presenting a complex of an HLA antigen and the present peptide on its surface and then isolating CTLs; or (4) introducing a gene including a polynucleotide encoding a T cell receptor (TCR) subunit binding to the peptide of this invention to the CTLs. The aforementioned APCs and exosomes can be prepared by methods described above and the method of (4) is detailed bellow in section “VIII. T cell receptor (TCR)”.

The CTLs of this invention can be derived from patients who are subject to treatment and/or prevention, and can be administered by themselves or in combination with other drugs including the peptides of this invention or exosomes for the purpose of regulating effects. The obtained CTLs act specifically against target cells presenting the peptides of this invention, for example, the same peptides used for induction. The target cells can be cells that endogenously express MYBL2, such as cancer cells, or cells that are transfected with the MYBL2 gene; and cells that present a peptide of this invention on the cell surface due to stimulation by the peptide can also serve as targets of activated CTL attack.

### VIII. T CELL RECEPTOR (TCR)

The present invention also provides a composition containing nucleic acids encoding polypeptides that are capable of forming a subunit of a T cell receptor (TCR), and methods of using the same. The TCR subunits have the ability to form TCRs that confer specificity to T cells against tumor cells expressing MYBL2. By using the known methods in the art, the nucleic acids of alpha- and beta-chains as the TCR subunits of the CTL induced with one or more peptides of this invention can be identified (WO2007/032255 and Morgan et al., J Immunol, 171, 3288 (2003)). For example, the PCR method is preferred to analyze the TCR. The PCR primers for the analysis can be, for example, 5′-R primers (5′-gtctaccaggcattcgcttcat-3′) as 5′ side primers (SEQ ID NO: 7) and 3-TRa-C primers (5′-tcagctggaccacagccgcagcgt-3′) specific to TCR alpha chain C region (SEQ ID NO: 8), 3-TRb-C1 primers (5′-tcagaaatcctttctcttgac-3′) specific to TCR beta chain C1 region (SEQ ID NO: 9) or 3-TRbeta-C2 primers (5′-ctagcctctggaatcctttctctt-3′) specific to TCR beta chain C2 region (SEQ ID NO: 10) as 3′ side primers, but not limited thereto. The derivative TCRs can bind target cells displaying the MYBL2 peptide with high avidity, and optionally mediate efficient killing of target cells presenting the MYBL2 peptide in vivo and in vitro.

The nucleic acids encoding the TCR subunits can be incorporated into suitable vectors, e.g., retroviral vectors. These vectors are well known in the art. The nucleic acids or the vectors containing them usefully can be transferred into a T cell, for example, a T cell from a patient. Advantageously, the invention provides an offthe-shelf composition allowing rapid modification of a patient's own T cells (or those of another mammal) to rapidly and easily produce modified T cells having excellent cancer cell killing properties.

The specific TCR is a receptor capable of specifically recognizing a complex of a peptide of the present invention and HLA molecule, giving a T cell specific activity against the target cell when the TCR on the surface of the T cell. A specific recognition of the above complex may be confirmed by any known methods, and preferred methods include, for example, tetramer analysis using HLA molecule and peptide of the invention, and ELISPOT assay. By performing the ELISPOT assay, it can be confirmed that a T cell expressing the TCR on the cell surface recognizes a cell by the TCR, and that the signal is transmitted intracellularly. The confirmation that the above-mentioned complex can give a T cell cytotoxic activity when the complex exists on the T cell surface may also be carried out by a known method. A preferred method includes, for example, the determination of cytotoxic activity against an HLA positive target cell, such as chromium release assay.

Also, the present invention provides CTLs which are prepared by transduction with the nucleic acids encoding the TCR subunits polypeptides that bind to the MYBL2 peptide of, e.g., SEQ ID NOs: 2-4, especially 3, in the context of HLA-A02. The transduced CTLs are capable of homing to cancer cells in vivo, and can be expanded by well known in vitro culturing methods (e.g., Kawakami et al., J. Immunol., 142, 3452-3461 (1989)). The CTLs of the invention can be used to form an immunogenic composition useful in treating or the prevention of cancer in a patient in need of therapy or protection (WO2006/031221).

### IX. PHARMACEUTICAL AGENTS OR COMPOSITIONS

Since MYBL2 expression is specifically elevated in cancers including testicular tumor, pancreatic cancer, bladder cancer, non-small cell lung cancer, small cell lung cancer and esophageal cancer, as compared with normal tissue, the peptides of the present invention or polynucleotides encoding such peptides can be used for the treatment and/or for the prophylaxis of cancer or tumor, and/or for prevention of postoperative recurrence thereof. Thus, the present invention provides a pharmaceutical agent or composition for treating and/or for the prophylaxis of cancer or tumor, and/or for prevention of postoperative recurrence thereof, such agent or composition including as an active ingredient one or more of the peptides of the present invention, or polynucleotides encoding the peptides. Alternatively, the present peptides can be expressed on the surface of any of the foregoing exosomes or cells, such as APCs for the use as pharmaceutical agents or compositions. In addition, the aforementioned CTLs which target any of the peptides of the invention can also be used as the active ingredient of the present pharmaceutical agents or compositions.

In another embodiment, the present invention also provides the use of an active ingredient selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention in manufacturing a pharmaceutical composition or agent for treating cancer or tumor.

Alternatively, the present invention further provides an active ingredient selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention for use in treating cancer of tumor.

Alternatively, the present invention further provides a method or process for manufacturing a pharmaceutical composition or agent for treating cancer or tumor, wherein the method or process includes the step of formulating a pharmaceutically or physiologically acceptable carrier with an active ingredient selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention as active ingredients.

In another embodiment, the present invention also provides a method or process for manufacturing a pharmaceutical composition or agent for treating cancer or tumor, wherein the method or process includes the steps of admixing an active ingredient with a pharmaceutically or physiologically acceptable carrier, wherein the active ingredient is selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention.

Alternatively, the pharmaceutical composition or agent or the present invention may be used for either or both the prophylaxis of cancer or tumor and prevention of postoperative recurrence thereof.

The present pharmaceutical agents or compositions find use as a vaccine. In the context of the present invention, the phrase “vaccine” (also referred to as an “immunogenic composition”) refers to a substance that has the function to induce anti-tumor immunity upon inoculation into animals.

The pharmaceutical agents or compositions of the present invention can be used to treat and/or prevent cancers or tumors, and/or prevention of postoperative recurrence thereof in subjects or patients including human and any other mammal including, but not limited to, mouse, rat, guinea-pig, rabbit, cat, dog, sheep, goat, pig, cattle, horse, monkey, baboon, and chimpanzee, particularly a commercially important animal or a domesticated animal.

According to the present invention, peptides having an amino acid sequence of any one of SEQ ID NOs: 2-4, especially 3, have been found to be HLA-A2 restricted epitope peptides or candidates that can induce potent and specific immune response. Therefore, the present pharmaceutical agents or compositions which include any of these peptides having the amino acid sequences of SEQ ID NOs: 2-4, especially 3, are particularly suited for the administration to subjects whose HLA antigen is HLA-A2. The same applies to pharmaceutical agents and compositions which include polynucleotides encoding any of these peptides (i.e., the polynucleotides of this invention).

Cancers or tumors to be treated by the pharmaceutical agents or compositions of the present invention are not limited and include all kinds of cancers or tumors wherein MYBL2 is involved (e.g., is overexpressed), including, for example, testicular tumor, pancreatic cancer, bladder cancer, non-small cell lung cancer, small cell lung cancer and esophageal cancer.

The present pharmaceutical agents or compositions can contain in addition to the aforementioned active ingredients, other peptides which have the ability to induce CTLs against cancerous cells, other polynucleotides encoding the other peptides, other cells that present the other peptides, or such. Herein, the other peptides that have the ability to induce CTLs against cancerous cells are exemplified by cancer specific antigens (e.g., identified TAAs), but are not limited thereto.

If needed, the pharmaceutical agents or compositions of the present invention can optionally include other therapeutic substances as an active ingredient, so long as the substance does not inhibit the antitumoral effect of the active ingredient, e.g., any of the present peptides. For example, formulations can include anti-inflammatory agents, pain killers, chemotherapeutics, and the like. In addition to including other therapeutic substances in the medicament itself, the medicaments of the present invention can also be administered sequentially or concurrently with the one or more other pharmacologic agents. The amounts of medicament and pharmacologic agent depend, for example, on what type of pharmacologic agent(s) is/are used, the disease being treated, and the scheduling and routes of administration.

It should be understood that in addition to the ingredients particularly mentioned herein, the pharmaceutical agents or compositions of this invention can include other agents conventional in the art having regard to the type of formulation in question.

In one embodiment of the present invention, the present pharmaceutical agents or compositions can be included in articles of manufacture and kits containing materials useful for treating the pathological conditions of the disease to be treated, e.g., cancer. The article of manufacture can include a container of any of the present pharmaceutical agents or compositions with a label. Suitable containers include bottles, vials, and test tubes. The containers can be formed from a variety of materials, such as glass or plastic. The label on the container should indicate the agent is used for treating or prevention of one or more conditions of the disease. The label can also indicate directions for administration and so on.

In addition to the container described above, a kit including a pharmaceutical agent or composition of the present invention can optionally further include a second container housing a pharmaceutically-acceptable diluent. It can further include other materials desirable from a commercial and user standpoint, including other buffers, diluents, filters, needles, syringes, and package inserts with instructions for use.

The pharmaceutical agents or compositions can, if desired, be presented in a pack or dispenser device which can contain one or more unit dosage forms containing the active ingredient. The pack can, for example, include metal or plastic foil, such as a blister pack. The pack or dispenser device can be accompanied by instructions for administration.

(1) Pharmaceutical Agents or Compositions Containing the Peptides as the Active Ingredient

The peptides of this invention can be administered directly as a pharmaceutical agent or composition, or if necessary, may be formulated by conventional formulation methods. In the latter case, in addition to the peptides of this invention, carriers, excipients, and such that are ordinarily used for drugs can be included as appropriate without particular limitations. Examples of such carriers are sterilized water, physiological saline, phosphate buffer, culture fluid and such. Furthermore, the pharmaceutical agents or compositions can contain as necessary, stabilizers, suspensions, preservatives, surfactants and such. The pharmaceutical agents or compositions of this invention can be used for anticancer purposes.

The peptides of this invention can be prepared as a combination composed of two or more of the peptides of the present invention, to induce CTLs in vivo. The peptide combination can take the form of a cocktail or can be conjugated to each other using standard techniques. For example, the peptides can be chemically linked or expressed as a single fusion polypeptide sequence. The peptides in the combination can be the same or different. By administering the peptides of this invention, the peptides are presented at a high density by the HLA antigens on APCs, then CTLs that specifically react toward the complex formed between the displayed peptide and the HLA antigen are induced. Alternatively, APCs (e.g., DCs) are removed from subjects and then stimulated by the peptides of the present invention to obtain APCs that present any of the peptides of this invention on their cell surface. These APCs are re-administered to the subjects to induce CTLs in the subjects, and as a result, aggressiveness towards the tumor-associated endothelium can be increased.

The pharmaceutical agents or compositions for the treatment and/or prevention of cancer or tumor, which include a peptide of this invention as the active ingredient, can also include an adjuvant known to effectively induce cellular immunity. Alternatively, the pharmaceutical agents or compositions can be administered with other active ingredients or administered by formulation into granules. An adjuvant refers to a compound that enhances the immune response against the protein when administered together (or successively) with the protein having immunological activity. Adjuvants contemplated herein include those described in the literature (Clin Microbiol Rev 1994, 7: 277-89). Example of suitable adjuvants include aluminum phosphate, aluminum hydroxide, alum, cholera toxin, salmonella toxin, Incomplete Freund's adjuvant (IFA), Complete Freund's adjuvant (CFA), ISCOMatrix, GM-CSF, CpG, 0/W emulsion, and such, but are not limited thereto.

Furthermore, liposome formulations, granular formulations in which the peptide is bound to few-micrometers diameter beads, and formulations in which a lipid is bound to the peptide may be conveniently used.

In another embodiment of the present invention, the peptides of the present invention may also be administered in the form of a pharmaceutically acceptable salt. Preferable examples of the salts include salts with an alkali metal, salts with a metal, salts with an organic base, salts with an organic acid and salts with an inorganic acid. As used herein, the phrase “pharmaceutically acceptable salt” refers to those salts that retain the biological effectiveness and properties of the compound and which are obtained by reaction with inorganic acids or bases such as hydrochloric acid, hydrobromic acid, sulfuric acid, nitric acid, phosphoric acid, methanesulfonic acid, ethanesulfonic acid, ptoluenesulfonic acid, salicylic acid and the like. Examples of preferred salts include salts with an alkali metal, salts with a metal, salts with an organic base, salts with an organic acid and salts with an inorganic acid.

In some embodiments, the pharmaceutical agents or compositions of the present invention may further include a component which primes CTLs. Lipids have been identified as agents capable of priming CTLs in vivo against viral antigens. For example, palmitic acid residues can be attached to the epsilon- and alpha-amino groups of a lysine residue and then linked to a peptide of the present invention. The lipidated peptide can then be administered either directly in a micelle or particle, incorporated into a liposome, or emulsified in an adjuvant. As another example of lipid priming of CTL responses, E. coli lipoproteins, such as tripalmitoylS-glycerylcysteinyl-seryl-serine (P3CSS) can be used to prime CTL when covalently attached to an appropriate peptide (see, e.g., Deres et al., Nature 1989, 342: 561-4).

The method of administration can be oral, intradermal, subcutaneous, intravenous injection, or such, and systemic administration or local administration to the vicinity of the targeted sites. The administration can be performed by single administration or boosted by multiple administrations. The dose of the peptides of this invention can be adjusted appropriately according to the disease to be treated, age of the patient, weight, method of administration, and such, and is ordinarily 0.001 mg to 1000 mg, for example, 0.1 mg to 10 mg, and can be administered once in a few days to few months. One skilled in the art can appropriately select a suitable dose.

(2) Pharmaceutical Agents or Compositions Containing Polynucleotides as the Active Ingredient

The pharmaceutical agents or compositions of the present invention can also contain nucleic acids encoding the peptides disclosed herein in an expressible form. Herein, the phrase “in an expressible form” means that the polynucleotide, when introduced into a cell, will be expressed in vivo as a polypeptide that induces anti-tumor immunity. In an exemplified embodiment, the nucleic acid sequence of the polynucleotide of interest includes regulatory elements necessary for expression of the polynucleotide. The polynucleotide(s) can be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The peptides of the invention can also be expressed by viral or bacterial vectors. Examples of expression vectors include attenuated viral hosts, such as vaccinia or fowlpox. This approach involves the use of vaccinia virus, e.g., as a vector to express nucleotide sequences that encode the peptide. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 1991, 351: 456-60. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al., Mol Med Today 2000, 6: 66-71; Shedlock et al., J Leukoc Biol 2000, 68: 793-806; Hipp et al., In Vivo 2000, 14: 571-85.

Delivery of a polynucleotide into a subject can be either direct, in which case the subject is directly exposed to a polynucleotide-carrying vector, or indirect, in which case, cells are first transformed with the polynucleotide of interest in vitro, then the cells are transplanted into the subject. These two approaches are known, respectively, as in vivo and ex vivo gene therapies.

For general reviews of the methods of gene therapy, see Goldspiel et al., Clinical Pharmacy 1993, 12: 488-505; Wu and Wu, Biotherapy 1991, 3: 87-95; Tolstoshev, Ann Rev Pharmacol Toxicol 1993, 33: 573-96; Mulligan, Science 1993, 260: 926-32; Morgan & Anderson, Ann Rev Biochem 1993, 62: 191-217; Trends in Biotechnology 1993, 11(5): 155-215). Methods commonly known in the art of recombinant DNA technology which can also be used for the present invention are described in eds. Ausubel et al., Current Protocols in Molecular Biology, John Wiley & Sons, NY, 1993; and Krieger, Gene Transfer and Expression, A Laboratory Manual, Stockton Press, NY, 1990.

The method of administration can be oral, intradermal, subcutaneous, intravenous injection, or such, and systemic administration or local administration to the vicinity of the targeted sites finds use. The administration can be performed by single administration or boosted by multiple administrations. The dose of the polynucleotide in the suitable carrier or cells transformed with the polynucleotide encoding the peptides of this invention can be adjusted appropriately according to the disease to be treated, age of the patient, weight, method of administration, and such, and is ordinarily 0.001 mg to 1000 mg, for example, 0.1 mg to 10 mg, and can be administered once every a few days to once every few months. One skilled in the art can appropriately select the suitable dose.

### X. METHODS USING THE PEPTIDES, EXOSOMES, APCS AND CTLS

The peptides and polynucleotides of the present invention can be used for inducing APCs and CTLs. The exosomes and APCs of the present invention can be also used for inducing CTLs. The peptides, polynucleotides, exosomes and APCs can be used in combination with any other compounds so long as the compounds do not inhibit their CTL inducibility. Thus, any of the aforementioned pharmaceutical agents or compositions of the present invention can be used for inducing CTLs, and in addition thereto, those including the peptides and polynucleotides can be also be used for inducing APCs as discussed explained below.

(1) Method of Inducing Antigen-Presenting Cells (APCs)

The present invention provides methods of inducing APCs with high CTL inducibility using the peptides or polynucleotides of this invention.

The methods of the present invention include the step of contacting APCs with the peptides of this invention in vitro, ex vivo or in vivo. For example, the method contacting APCs with the peptides ex vivo can include the steps of:

a: collecting APCs from a subject: and

b: contacting the APCs of step a with the peptide.

The APCs are not limited to a particular kind of cells and include DCs, Langerhans cells, macrophages, B cells, and activated T cells, which are known to present proteinaceous antigens on their cell surface so as to be recognized by lymphocytes. DCs can be preferably used due to its strongest CTL inducibility among the APCs. Any peptides of the present invention can be used as the peptide of step b by themselves or in combination with other peptides of this invention.

Alternatively, the peptides of the present invention may be administered to a subject to contact the peptides with APCs in vivo. Consequently, APCs with high CTL inducibility can be induced in the body of the subject. Thus, the present invention also contemplates a method of administering the peptides of this invention to a subject to induce APCs in vivo. It is also possible to administer polynucleotides encoding the peptides of this invention to a subject in an expressible form, so that the peptides of this invention are expressed and contacted with APCs in vivo, to consequently induce APCs with high CTL inducibility in the body of the subject. Thus, the present invention also contemplates a method of administering the polynucleotides of this invention to a subject to induce APCs in vivo. The phrase “expressible form” is defined above in section “IX. Pharmaceutical agents or compositions (2) Pharmaceutical agents containing polynucleotides as the active ingredient”.

Furthermore, the present invention includes introducing the polynucleotide of this invention into an APC to induce APCs with CTL inducibility. For example, the method may include the steps of:

a: collecting APCs from a subject: and

b: introducing a polynucleotide encoding a peptide of this invention.

Step b can be performed as described above in section “VI. Antigen-presenting cells”.

(2) Method of Inducing CTLs

The present invention also provides methods for inducing CTLs using the peptides, polynucleotides, or exosomes or APCs of this invention.

The present invention also provides methods for inducing CTLs using a polynucleotide encoding a polypeptide that is capable of forming a T cell receptor (TCR) subunit recognizing a complex of the peptides of the present invention and HLA antigens. Preferably, the methods for inducing CTLs include at least one step selected from among:

a) contacting a CD8-positive T cell with an antigen-presenting cell and/or an exosome that presents on its surface a complex of an HLA antigen and a peptide of the preset invention; and

b) introducing a polynucleotide encoding a polypeptide that is capable of forming a TCR subunit recognizing a complex of a peptide of the present invention and an HLA antigen into a CD8 positive cell.

When the peptides, the polynucleotides, APCs, or exosomes of the present invention are administered to a subject, CTLs are induced in the body of the subject, and the strength of the immune response targeting the cancer cells is enhanced. Thus, the present invention also contemplates a method which includes the step of administering the peptides, the polynucleotides, the APCs or exosomes of this invention to a subject to induce CTLs.

Alternatively, CTLs can be also induced by their ex vivo use. In such case, after the induction of CTLs, the activated CTLs would be returned to the subject. For example, a method of the present invention to induce CTLs can include steps of:

a) collecting APCs from a subject;

b) contacting the APCs of step a) with the peptide; and

c) co-culturing the APCs of step b with CD8-positive cells.

The APCs to be co-cultured with the CD8-positive cells in above step c can also be prepared by transferring a gene that includes a polynucleotide of this invention into APCs as described above in section “VI. Antigen-presenting cells”, though the present invention is not limited thereto and encompasses any APC that effectively presents on its surface a complex of an HLA antigen and a peptide of this invention.

Instead of such APCs, the exosomes that presents on its surface a complex of an HLA antigen and the peptide of this invention can be also used. Namely, the present invention also contemplates a method wherein exosomes presenting on its surface a complex of an HLA antigen and the peptide of this invention are co-cultured with CD8-positive cells. Such exosomes may be prepared by the methods described above in section “V. Exosomes”.

Furthermore, CTL can be induced by introducing a gene that includes a polynucleotide encoding the TCR subunit binding to the peptide of this invention into CD8-positive cells. Such transduction can be performed as described above in section “VIII. T cell receptor (TCR)”.

In addition, the present invention provides a method or process for manufacturing a pharmaceutical agent or composition inducing CTLs, wherein the method includes the step of admixing or formulating the peptide of the present invention with a pharmaceutically acceptable carrier.

(3) Method of Inducing Immune Response

Moreover, the present invention provides methods for inducing an immune response against diseases related to MYBL2. Suitable disease include cancer, examples of which include, but are not limited to, testicular tumor, pancreatic cancer, bladder cancer, non-small cell lung cancer, small cell lung cancer and esophageal cancer. The methods of the present invention include the step of administering agents or compositions containing any of the peptides of the present invention or polynucleotides encoding them. The present inventive method also contemplates the administration of exosomes or APCs presenting any of the peptides of the present invention. For details, see the item of “IX. Pharmaceutical agents or compositions”, particularly the part describing the use of the pharmaceutical agents and compositions of the present invention as vaccines. In addition, the exosomes and APCs that can be employed for the present methods for inducing immune response are described in detail under the items of “V. Exosomes”, “VI. Antigen-presenting cells (APCs)”, and (1) and (2) of “X. Methods using the peptides, exosomes, APCs and CTLs”, supra.

The present invention further provides methods for inducing an immune response against cancer, such as testicular tumor, pancreatic cancer, bladder cancer, non-small cell lung cancer, small cell lung cancer and esophageal cancer, in a subject. To that end, the present invention encompasses methods for manufacturing a pharmaceutical agent, substance or composition for inducing immune response, wherein the method may include the step of admixing or formulating a peptide or polynucleotide of the present invention with a pharmaceutically acceptable carrier. Alternatively, the method for inducing immune response may involve the step of formulating and administering a vaccine of the present invention, such vaccine including:

(a) one or more epitope peptides of the present invention, or an immunologically active fragment thereof;

(b) one or more polynucleotides encoding the epitope peptides or the immunologically active fragment of (a);

(c) one or more isolated CTLs of the present invention; or

(d) one or more isolated antigen-presenting cells of the present invention.

In the context of the present invention, a cancer overexpressing MYBL2 can be treated with these active ingredients. Examples of such cancers include, but are not limited to, testicular tumor, pancreatic cancer, bladder cancer, non-small cell lung cancer, small cell lung cancer and esophageal cancer. Accordingly, prior to the administration of the vaccines or pharmaceutical compositions including the active ingredients, it is preferable to confirm whether the expression level of MYBL2 in the cancer cells or tissues to be treated is enhanced compared with normal cells of the same organ. Thus, in one embodiment, the present invention provides a method for treating cancer (over)expressing MYBL2, which method may include the steps of:

i) determining the expression level of MYBL2 in cancer cells or tissue(s) obtained from a subject with the cancer to be treated;

ii) comparing the expression level of MYBL2 with normal control; and

iii) administrating at least one component selected from among (a) to (d) described above to a subject with cancer overexpressing MYBL2 compared with normal control.

Alternatively, the present invention also provides a vaccine or pharmaceutical composition that includes at least one component selected from among (a) to (d) described above, for use in administrating to a subject having cancer overexpressing MYBL2. In other words, the present invention further provides methods for identifying a subject to be treated with the MYBL2 polypeptide of the present invention, such methods including the step of determining an expression level of MYBL2 in subject-derived cancer cells or tissue(s), wherein an increase of the level compared to a normal control level of the gene indicates that the subject has cancer which may be treated with the MYBL2 polypeptide of the present invention. The methods of treating cancer of the present invention are described in more detail below.

A subject to be treated by the present method is preferably a mammal. Exemplary mammals include, but are not limited to, e.g., human, non-human primate, mouse, rat, dog, cat, horse, and cow.

According to the present invention, the expression level of MYBL2 in cancer cells or tissues obtained from a subject is determined. The expression level can be determined at the transcription (nucleic acid) product level, using methods known in the art. For example, the mRNA of MYBL2 may be quantified using probes by hybridization methods (e.g., Northern hybridization). The detection may be carried out on a chip or an array. The use of an array is preferable for detecting the expression level of MYBL2. Those skilled in the art can prepare such probes utilizing the sequence information of MYBL2. For example, the cDNA of MYBL2 may be used as the probes. If necessary, the probes may be labeled with a suitable label, such as dyes, fluorescent substances and isotopes, and the expression level of the gene may be detected as the intensity of the hybridized labels.

Any subject-derived cell or tissue can be used for the determination of MYBL2 expression so long as it includes the objective transcription or translation product of MYBL2. Examples of suitable samples include, but are not limited to, bodily tissues and fluids, such as blood, sputum and urine. Preferably, the subject-derived cell or tissue sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the subjected-derived sample. A transcription product of MYBL2 may also be quantified using primers by amplification-based detection methods (e.g., RT-PCR). Such primers may be prepared based on the available sequence information of the gene.

Specifically, a probe or primer used for the present method hybridizes under stringent, moderately stringent, or low stringent conditions to the mRNA of MYBL2. As used herein, the phrase “stringent (hybridization) conditions” refers to conditions under which a probe or primer will hybridize to its target sequence, but not to other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under a defined ionic strength, pH and nucleic acid concentration) at which 50% of the probes complementary to their target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

The probes or primers may be of specific sizes. The sizes may range from at least 10 nucleotides, at least 12 nucleotides, at least 15 nucleotides, at least 20 nucleotides, at least 25 nucleotides, at least 30 nucleotides and the probes and primers may range in size from 5-10 nucleotides, 10-15 nucleotides, 15-20 nucleotides, 20-25 nucleotides and 25-30 nucleotides.

Alternatively, the translation product may be detected for the diagnosis of the present invention. For example, the quantity of MYBL2 protein (SEQ ID NO: 6) may be determined. Methods for determining the quantity of the protein as the translation product include immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment or modified antibody retains the binding ability to the MYBL2 protein. Methods to prepare these kinds of antibodies for the detection of proteins are well known in the art, and any method may be employed in the present invention to prepare such antibodies and equivalents thereof.

As another method to detect the expression level of MYBL2 gene based on its translation product, the intensity of staining may be measured via immunohistochemical analysis using an antibody against the MYBL2 protein. Namely, in this measurement, strong staining indicates increased presence/level of the protein and, at the same time, high expression level of MYBL2 gene.

The expression level of a target gene, e.g., the MYBL2 gene, in cancer cells can be determined to be increased if the level increases from the control level (e.g., the level in normal cells) of the target gene by, for example, 10%, 25%, or 50%; or increases to more than 1.1 fold, more than 1.5 fold, more than 2.0 fold, more than 5.0 fold, more than 10.0 fold, or more.

The control level may be determined at the same time as the cancer cells by using a sample(s) previously collected and stored from a subject/subjects whose disease state(s) (cancerous or non-cancerous) is/are known. In addition, normal cells obtained from non-cancerous regions of an organ that has the cancer to be treated may be used as normal control. Alternatively, the control level may be determined by a statistical method based on the results obtained by analyzing previously determined expression level(s) of MYBL2 gene in samples from subjects whose disease states are known. Furthermore, the control level can be derived from a database of expression patterns from previously tested cells. Moreover, according to an aspect of the present invention, the expression level of MYBL2 gene in a biological sample may be compared to multiple control levels, determined from multiple reference samples. It is preferred to use a control level determined from a reference sample derived from a tissue type similar to that of the subject-derived biological sample. Moreover, it is preferred to use the standard value of the expression levels of MYBL2 gene in a population with a known disease state. The standard value may be obtained by any method known in the art. For example, a range of mean+/−2 S.D. or mean+/−3 S.D. may be used as the standard value.

In the context of the present invention, a control level determined from a biological sample that is known to be non-cancerous is referred to as a “normal control level”. On the other hand, if the control level is determined from a cancerous biological sample, it is referred to as a “cancerous control level”. Difference between a sample expression level and a control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

When the expression level of MYBL2 gene is increased as compared to the normal control level, or is similar/equivalent to the cancerous control level, the subject may be diagnosed with cancer to be treated.

The present invention also provides a method of (i) diagnosing or identifying whether a subject has the cancer to be treated, and/or (ii) selecting a subject for cancer treatment, which method includes the steps of:

a) determining the expression level of MYBL2 in cancer cells or tissue(s) obtained from a subject who is suspected to have the cancer to be treated;

b) comparing the expression level of MYBL2 with a normal control level;

c) diagnosing the subject as having the cancer to be treated, if the expression level of MYBL2 is increased as compared to the normal control level; and

d) selecting or identifying the subject for cancer treatment, if the subject is diagnosed as having the cancer to be treated, in step c).

Alternatively, such a method includes the steps of:

a) determining the expression level of MYBL2 in cancer cells or tissue(s) obtained from a subject who is suspected to have the cancer to be treated;

b) comparing the expression level of MYBL2 with a cancerous control level;

c) diagnosing the subject as having the cancer to be treated, if the expression level of MYBL2 is similar or equivalent to the cancerous control level; and

d) selecting or identifying the subject for cancer treatment, if the subject is diagnosed as having the cancer to be treated, in step c).

The present invention also provides a kit for determining a subject suffering from cancer that can be treated with the MYBL2 polypeptide of the present invention, which may also be useful in assessing and/or monitoring the efficacy of a cancer immunotherapy. Preferably, the cancer includes, but is not limited to, testicular tumor, pancreatic cancer, bladder cancer, non-small cell lung cancer, small cell lung cancer and esophageal cancer. More particularly, the kit preferably includes at least one reagent for detecting the expression of the MYBL2 gene in a subject-derived cancer cell, which reagent may be selected from the group of:

(a) a reagent for detecting mRNA of the MYBL2 gene;

(b) a reagent for detecting the MYBL2 protein; and

(c) a reagent for detecting the biological activity of the MYBL2 protein.

Examples of reagents suitable for detecting mRNA of the MYBL2 gene include nucleic acids that specifically bind to or identify the MYBL2 mRNA, such as oligonucleotides which have a complementary sequence to a portion of the MYBL2 mRNA. These kinds of oligonucleotides are exemplified by primers and probes that are specific to the MYBL2 mRNA. These kinds of oligonucleotides may be prepared based on methods well known in the art. If needed, the reagent for detecting the MYBL2 mRNA may be immobilized on a solid matrix. Moreover, more than one reagent for detecting the MYBL2 mRNA may be included in the kit.

On the other hand, examples of reagents suitable for detecting the MYBL2 protein include antibodies to the MYBL2 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment or modified antibody retains the binding ability to the MYBL2 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of the antibodies to their targets are well known in the art, and any labels and methods may be employed for the present invention. Moreover, more than one reagent for detecting the MYBL2 protein may be included in the kit.

The kit may contain more than one of the aforementioned reagents. For example, tissue samples obtained from subjects without cancer or suffering from cancer, may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

In an embodiment of the present invention, when the reagent is a probe against the MYBL2 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of a test sample, the number of sites displaying a detectable signal provides a quantitative indication of the amount of MYBL2 mRNA present in the sample. The detection sites may be configured in any suitably detectable shape and are typically in the shape of a bar or dot spanning the width of a test strip.

The kit of the present invention may further include a positive control sample or MYBL2 standard sample. The positive control sample of the present invention may be prepared by collecting MYBL2 positive samples and then assaying their MYBL2 levels. Alternatively, a purified MYBL2 protein or polynucleotide may be added to cells that do not express MYBL2 to form the positive sample or the MYBL2 standard sample. In the present invention, purified MYBL2 may be a recombinant protein. The MYBL2 level of the positive control sample is, for example, more than the cut off value.

In one embodiment, the present invention further provides a diagnostic kit including, a protein or a partial protein thereof capable of specifically recognizing the antibody of the present invention or the fragment thereof.

Examples of the partial peptide of the protein of the present invention include polypeptides consisting of at least 8, preferably 15, and more preferably 20 contiguous amino acids in the amino acid sequence of the protein of the present invention. Cancer can be diagnosed by detecting an antibody in a sample (e.g., blood, tissue) using a protein or a peptide (polypeptide) of the present invention. The method for preparing the protein of the present invention and peptides are as described above.

Diagnostic method for cancer can be done by determining the difference between the amount of anti-MYBL2 antibody and that in the corresponding control sample as described above. The subject is suspected to be suffering from cancer, if cells or tissues of the subject contain antibodies against the expression products (MYBL2) of the gene and the quantity of the anti-MYBL2 antibody is determined to be more than the cut off value in level compared to that in normal control.

In another embodiment, a diagnostic kit of the present invention may include the peptide of the present invention and an HLA molecule binding thereto. The method for detecting antigen specific CTLs using antigenic peptides and HLA molecules has already been established (for example, Altman J D et al., Science. 1996, 274(5284): 94-6). Thus, the complex of the peptide of the present invention and the HLA molecule can be applied to the detection method to detect tumor antigen specific CTLs, thereby enabling earlier detection, recurrence and/or metastasis of cancer. Further, it can be employed for the selection of subjects applicable with the pharmaceuticals including the peptide of the present invention as an active ingredient, or the assessment of the treatment effect of the pharmaceuticals.

Particularly, according to the known method (see, for example, Altman J D et al., Science. 1996, 274(5284): 94-6), the oligomermultimer complex, such as tetramer, of the radiolabeled HLA molecule and the peptide of the present invention can be prepared. With using the complex, the diagnosis can be done, for example, by quantifying the antigen-peptide specific CTLs in the peripheral blood lymphocytes derived from the subject suspected to be suffering from cancer.

The present invention further provides a method or diagnostic agents for evaluating immunological response of a subject by using peptide epitopes as described herein. In one embodiment of the invention, HLA-A-2 restricted peptides as described herein are used as reagents for evaluating or predicting an immune response of a subject. The immune response to be evaluated is induced by contacting an immunogen with immunocompetent cells in vitro or in vivo. In preferred embodiments, the immunocompetent cells for evaluating an immunological response, may be selected among peripheral blood, peripheral blood lymphocyte (PBL), and peripheral blood mononuclear cell (PBMC). Methods for collecting or isolating such immunocompetent cells are well known in the arts. In some embodiments, any agent that may result in the production of antigen specific CTLs that recognize and bind to the peptide epitope(s) may be employed as the reagent. The peptide reagent need not be used as the immunogen. Assay systems that are used for such an analysis include relatively recent technical developments such as tetramers, staining for, intracellular lymphokines and interferon release assays, or ELISPOT assays. In a preferred embodiment, immunocompetent cells to be contacted with peptide reagent may be antigen presenting cells including dendritic cells.

For example, peptides of the present invention may be used in tetramer multimer staining assays to assess peripheral blood mononuclear cells for the presence of antigen-specific CTLs following exposure to a tumor cell antigen or an immunogen. The HLA tetrameric multimeric complex may be used to directly visualize antigen specific CTLs (see, e.g., Ogg et al., Science 279: 2103-2106, 1998; and Altman et al, Science 174: 94-96, 1996) and determine the frequency of the antigen-specific CTL population in a sample of peripheral blood mononuclear cells. A reagent of tetramer multimerreagent such as tetramer using a peptide of the invention may be generated as follows: described below.

A peptide that binds to an HLA molecule is refolded in the presence of the corresponding HLA heavy chain and beta 2-microglobulin to generate a trimolecular complex. In the complex, carboxyl terminal of the heavy chain is biotinylated at a site that was previously engineered into the protein. Then, streptavidin is added to the complex to form tetramer composed of the trimolecular complex and streptavidin. By means of fluorescently labeled streptavidin, the tetramer can be used to stain antigen-specific cells. The cells can then be identified, for example, by flow cytometry. Such an analysis may be used for diagnostic or prognostic purposes. Cells identified by the procedure can also be used for therapeutic purposes.

The present invention also provides reagents to evaluate immune recall responses (see, e.g., Bertoni et al., J. Clin. Invest. 100: 503-513, 1997 and Penna et al., J. Exp. Med. 174: 1565-1570, 1991) comprising peptides of the present invention. For example, patient PBMC samples from individuals with cancer to be treated are analyzed for the presence of antigen-specific CTLs using specific peptides. A blood sample containing mononuclear cells can be evaluated by cultivating the PBMCs and stimulating the cells with a peptide of the invention. After an appropriate cultivation period, the expanded cell population can be analyzed, for example, for CTL activity. The peptides may be also used as reagents to evaluate the efficacy of a vaccine. PBMCs obtained from a patient vaccinated with an immunogen may be analyzed using, for example, either of the methods described above. The patient is HLA typed, and peptide epitope reagents that recognize the allele specific molecules present in that patient are selected for the analysis. The immunogenicity of the vaccine may be indicated by the presence of epitope-specific CTLs in the PBMC sample. The peptides of the invention may be also used to make antibodies, using techniques well known in the art (see, e.g. CURRENT PROTOCOLS IN IMMUNOLOGY, Wiley/Greene, NY; and Antibodies A Laboratory Manual, Harlow and Lane, Cold Spring Harbor Laboratory Press, 1989), which may be useful as reagents to diagnose or monitor cancer. Such antibodies may include those that recognize a peptide in the context of an HLA molecule, i.e., antibodies that bind to a peptide-MHC complex.

Alternatively, the invention also provides a number of uses, some of which are described herein. For instance, the present invention provides a method for diagnosing or detecting a disorder characterized by expression of a MYBL2 immunogenic polypeptide. These methods involve determining expression of a MYBL2 HLA binding peptide, or a complex of a MYBL2 HLA binding peptide and an HLA class I molecule in a biological sample. The expression of a peptide or complex of peptide and HLA class I molecule can be determined or detected by assaying with a binding partner for the peptide or complex. In an preferred embodiment, a binding partner for the peptide or complex is an antibody that recognizes and specifically bind to the peptide. The expression of MYBL2 in a biological sample, such as a tumor biopsy, can also be tested by standard PCR amplification protocols using MYBL2 primers. An example of tumor expression is presented herein and further disclosure of exemplary conditions and primers for MYBL2 amplification can be found in WO2003/27322.

Preferably, the diagnostic methods involve contacting a biological sample isolated from a subject with an agent specific for the MYBL2 HLA binding peptide to detect the presence of the MYBL2 HLA binding peptide in the biological sample. As used herein, “contacting” means placing the biological sample in sufficient proximity to the agent and under the appropriate conditions of, e.g., concentration, temperature, time, ionic strength, to allow the specific interaction between the agent and MYBL2 HLA binding peptide that are present in the biological sample. In general, the conditions for contacting the agent with the biological sample are conditions known by those of ordinary skill in the art to facilitate a specific interaction between a molecule and its cognate (e.g., a protein and its receptor cognate, an antibody and its protein antigen cognate, a nucleic acid and its complementary sequence cognate) in a biological sample. Exemplary conditions for facilitating a specific interaction between a molecule and its cognate are described in U.S. Pat. No. 5,108,921, issued to Low et al.

The diagnostic method of the present invention can be performed in either or both of in vivo and in vitro. Accordingly, biological sample can be located in vivo or in vitro in the present invention. For example, the biological sample can be a tissue in vivo and the agent specific for the MYBL2 immunogenic polypeptide can be used to detect the presence of such molecules in the tissue. Alternatively, the biological sample can be collected or isolated in vitro (e.g., a blood sample, tumor biopsy, tissue extract). In a particularly preferred embodiment, the biological sample can be a cell-containing sample, more preferably a sample containing tumor cells collected from a subject to be diagnosed or treated.

Alternatively, the diagnosis can be done, by a method which allows direct quantification of antigen-specific T cells by staining with Fluorescein-labelled HLA multimeric complexes (for example, Altman, J. D. et al., 1996, Science 274: 94; Altman, J. D. et al., 1993, Proc. Natl. Acad. Sci. USA 90: 10330). Staining for intracellular lymphokines, and interferon-gamma release assays or ELISPOT assays also has been provided. Tetramer Multimer staining, intracellular lymphokine staining and ELISPOT assays all appear to be at least 10-fold more sensitive than more conventional assays (Murali-Krishna, K. et al., 1998, Immunity 8: 177; Lalvani, A. et al., 1997, J. Exp. Med. 186: 859; Dunbar, P. R. et al., 1998, Curr. Biol. 8: 413). Pentamers (e.g., US 2004-209295A), dextramers (e.g., WO 02/072631), and streptamers (e.g., Nature medicine 6. 631-637 (2002)) may also be used.

For instance, in some embodiments, the present invention provides a method for diagnosing or evaluating an immunological response of a subject administered at least one of MYBL2 peptides of the present invention, the method including the steps of:

(a) contacting an immunogen with immunocompetent cells under the condition suitable of induction of CTL specific to the immunogen;

(b) detecting or determining induction level of the CTL induced in step (a); and

(c) correlating the immunological response of the subject with the CTL induction level.

In the present invention, the immunogen is at least one of (a) a MYBL2 peptide selected from among the amino acid sequences of SEQ ID NOs: 2 to 4, peptides having such amino acid sequences, and peptides having in which such amino acid sequences have been modified with 1, 2 or more amino acid substitution(s). In the meantime, conditions suitable of induction of immunogen specific CTL are well known in the art. For example, immunocompetent cells may be cultured in vitro under the presence of immunogen(s) to induce immunogen specific CTL. In order to induce immunogen specific CTLs, any stimulating factors may be added to the cell culture. For example, IL-2 is preferable stimulating factors for the CTL induction.

In some embodiments, the step of monitoring or evaluating immunological response of a subject to be treated with peptide cancer therapy may be performed before, during and/or after the treatment. In general, during a protocol of cancer therapy, immunogenic peptides are administered repeatedly to a subject to be treated. For example, immunogenic peptides may be administered every week for 3-10 weeks. Accordingly, the immunological response of the subject can be evaluated or monitored during the cancer therapy protocol. Alternatively, the step of evaluation or monitoring of immunological response to the cancer therapy may at the completion of the therapy protocol.

According to the present invention, enhanced induction of immunogen specific CTL as compared with a control indicates that the subject to be evaluated or diagnosed immunologically responded to the immunogen(s) which have been administered. Suitable controls for evaluating the immunological response may include, for example, a CTL induction level when the immunocompetent cells are contacted with no peptide, or control peptide(s) having amino acid sequences other than any MYBL2 peptides. (e.g. random amino acid sequence).

In a preferred embodiment, the immunological response of the subject is evaluated in a sequence specific manner, by comparison with an immunological response between each immunogen administered to the subject. In particular, even when a mixture of some kinds of MYBL2 peptides is administered to the subject, immunological response might vary depending on the peptides. In that case, by comparison of the immunological response between each peptide, peptides to which the subject show higher response can be identified.

### XI. ANTIBODIES

The present invention provides antibodies that bind to the peptide of the present invention. Preferred antibodies specifically bind to the peptide of the present invention and will not bind (or will bind weakly) to non-peptide of the present invention. Alternatively, antibodies bind the peptide of the invention as well as the homologs thereof.

Antibodies against the peptide of the invention can find use in cancer diagnostic and prognostic assays, and imaging methodologies. Similarly, such antibodies can find use in the treatment, diagnosis, and/or prognosis of other cancers, to the extent MYBL2 is also expressed or overexpressed in cancer patient. Moreover, intracellularly expressed antibodies (e.g., single chain antibodies) are therapeutically useful in treating cancers in which the expression of MYBL2 is involved, examples of which include, but are not limited to breast cancer, cervical cancer, colorectal cancer, esophageal cancer, gastric cancer, gastric diffuse-type cancer, lymphoma, neuroblastoma, pancreatic cancer.

The present invention also provides various immunological assay for the detection and/or quantification of the MYBL2 protein (SEQ ID NO: 6) or fragments thereof including polypeptides having an amino acid sequence selected from among SEQ ID NOs: 2 to 4. Such assays can comprise one or more anti-MYBL2 antibodies capable of recognizing and binding a MYBL2 protein or fragments thereof, as appropriate. In the context of the present invention, anti-MYBL2 antibodies binding to MYBL2 polypeptide preferably recognize a polypeptide having an amino acid sequence selected from among SEQ ID NOs: 2 to 4. A binding specificity of antibody can be confirmed with inhibition test. That is, when the binding between an antibody to be analyzed and full-length of MYBL2 polypeptide was inhibited under presence of any fragment polypeptides having an amino acid sequence selected from among SEQ ID NOs: 2 to 4, it is shown that this antibody specifically binds to the fragment. In the context of the present invention, such immunological assays are performed within various immunological assay formats well known in the art, including but not limited to various types of radioimmunoassays, immuno-chromatgraph technique, enzyme-linked immunosorbent assays (ELISA), enzyme-linked immunofluorescent assays (ELIFA), and the like.

Related immunological but non-antibody assays of the invention also comprise T cell immunogenicity assays (inhibitory or stimulatory) as well as major histocompatibility complex (MHC) binding assays. In addition, immunological imaging methods capable of detecting cancers expressing MYBL2 are also provided by the invention, including but not limited to radioscintigraphic imaging methods using labeled antibodies of the present invention. Such assays are clinically useful in the detection, monitoring, and prognosis of MYBL2 expressing cancers, examples of which include, but are not limited to, breast cancer, cervical cancer, colorectal cancer, esophageal cancer, gastric cancer, gastric diffuse-type cancer, lymphoma, neuroblastoma, pancreatic cancer.

The present invention provides an antibody that binds to the peptide of the invention. The antibody of the invention can be used in any form, such as monoclonal or polyclonal antibodies, and includes antiserum obtained by immunizing an animal such as a rabbit with the peptide of the invention, all classes of polyclonal and monoclonal antibodies, human antibodies and humanized antibodies produced by genetic recombination.

A peptide of the invention used as an antigen to obtain an antibody may be derived from any animal species, but preferably is derived from a mammal such as a human, mouse, or rat, more preferably from a human. A human-derived peptide may be obtained from the nucleotide or amino acid sequences disclosed herein.

According to the present invention, the peptide to be used as an immunization antigen may be a complete protein or a partial peptide of the protein. A partial peptide may comprise, for example, the amino (N)-terminal or carboxy (C)-terminal fragment of a peptide of the present invention.

Herein, an antibody is defined as a protein that reacts with either the full length or a fragment of a MYBL2 peptide. In a preferred embodiment, an antibody of the present invention recognizes fragment peptides of MYBL2 having an amino acid sequence selected from among SEQ ID NOs: 2 to 4. Methods for synthesizing oligopeptide are well known in the arts. After the synthesis, peptides may be optionally purified prior to use as immunogen. In the present invention, the oligopeptide (e.g. 9 or 10 mer) may be conjugated or linked with carriers to enhance the immunogenicity. Keyhole-limpet hemocyanin (KLH) is well known as the carrier. Method for conjugating KLH and peptide are also well known in the arts.

Alternatively, a gene encoding a peptide of the invention or its fragment may be inserted into a known expression vector, which is then used to transform a host cell as described herein. The desired peptide or its fragment may be recovered from the outside or inside of host cells by any standard method, and may subsequently be used as an antigen. Alternatively, whole cells expressing the peptide or their lysates or a chemically synthesized peptide may be used as the antigen.

Any mammalian animal may be immunized with the antigen, but preferably the compatibility with parental cells used for cell fusion is taken into account. In general, animals of Rodentia, Lagomorpha or Primates are used. Animals of the family Rodentia include, for example, mouse, rat and hamster. Animals of the family Lagomorpha include, for example, rabbit. Animals of the Primate families include, for example, a monkey of Catarrhini (old world monkey) such as Macaca fascicularis, rhesus monkey, sacred baboon and chimpanzees.

Methods for immunizing animals with antigens are known in the art. Intraperitoneal injection or subcutaneous injection of antigens is a standard method for the immunization of mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion and then administered to mammalian animals. Preferably, it is followed by several administrations of antigen mixed with an appropriately amount of Freund's incomplete adjuvant every 4 to 21 days. An appropriate carrier may also be used for immunization. After immunization as above, serum is examined by a standard method for an increase in the amount of desired antibodies.

Polyclonal antibodies against the peptides of the present invention may be prepared by collecting blood from the immunized mammal examined for the increase of desired antibodies in the serum, and by separating serum from the blood by any conventional method. Polyclonal antibodies may include serum containing the polyclonal antibodies, as well as the fraction containing the polyclonal antibodies that may be isolated from the serum. Immunoglobulin G or M can be prepared from a fraction which recognizes only the peptide of the present invention using, for example, an affinity column coupled with the peptide of the present invention, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al. (Galfre and Milstein, Methods Enzymol 73: 3-46 (1981)).

Resulting hybridomas obtained by the cell fusion may be selected by cultivating them in a standard selection medium, such as HAT medium (hypoxanthine, aminopterin and thymidine containing medium). The cell culture is typically continued in the HAT medium for several days to several weeks, the time being sufficient to allow all the other cells, with the exception of the desired hybridoma (non-fused cells), to die. Then, the standard limiting dilution is performed to screen and clone a hybridoma cell producing the desired antibody.

In addition to the above method, in which a non-human animal is immunized with an antigen for preparing hybridoma, human lymphocytes such as those infected by EB virus may be immunized with a peptide, peptide expressing cells or their lysates in vitro. Then, the immunized lymphocytes are fused with human-derived myeloma cells that are capable of indefinitely dividing, such as U266, to yield a hybridoma producing a desired human antibody that is able to bind to the peptide can be obtained (Unexamined Published Japanese Patent Application No. (JP-A) Sho 63-17688).

The obtained hybridomas are subsequently transplanted into the abdominal cavity of a mouse and the ascites are extracted. The obtained monoclonal antibodies can be purified by, for example, ammonium sulfate precipitation, a protein A or protein G column, DEAE ion exchange chromatography or an affinity column to which the peptide of the present invention is coupled. The antibody of the present invention can be used not only for purification and detection of the peptide of the present invention, but also as a candidate for agonists and antagonists of the peptide of the present invention.

Alternatively, an immune cell, such as an immunized lymphocyte, producing antibodies may be immortalized by an oncogene and used for preparing monoclonal antibodies.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody. The present invention also provides recombinant antibodies prepared as described above.

Furthermore, an antibody of the present invention may be a fragment of an antibody or modified antibody, so long as it binds to one or more of the peptides of the invention. For instance, the antibody fragment may be Fab, F(ab′)2, Fv or single chain Fv (scFv), in which Fv fragments from H and L chains are ligated by an appropriate linker (Huston et al., Proc Natl Acad Sci USA 85: 5879-83 (1988)). More specifically, an antibody fragment may be generated by treating an antibody with an enzyme, such as papain or pepsin. Alternatively, a gene encoding the antibody fragment may be constructed, inserted into an expression vector and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

An antibody may be modified by conjugation with a variety of molecules, such as polyethylene glycol (PEG). The present invention provides for such modified antibodies. The modified antibody can be obtained by chemically modifying an antibody. These modification methods are conventional in the field.

Alternatively, an antibody of the present invention may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) and the constant region derived from human antibody. Such antibodies can be prepared according to known technology. Humanization can be performed by substituting rodent CDRs or CDR sequences for the corresponding sequences of a human antibody (see e.g., Verhoeyen et al., Science 239:1534-1536 (1988)). Accordingly, such humanized antibodies are chimeric antibodies, wherein substantially less than an intact human variable domain has been substituted by the corresponding sequence from a non-human species.

Fully human antibodies comprising human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom & Winter, J. Mol. Biol. 227:381 (1991)), Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

Antibodies obtained as above may be purified to homogeneity. For example, the separation and purification of the antibody can be performed according to the separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis and isoelectric focusing (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS and Sepharose F.F. (Pharmacia). Exemplary chromatography, with the exception of affinity includes, for example, ion-exchange chromatography, hydrophobic chromatography, gel filtration, reverse-phase chromatography, adsorption chromatography and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

For example, measurement of absorbance, enzyme-linked immunosorbent assay (ELISA), enzyme immunoassay (EIA), radioimmunoassay (RIA) and/or immunofluorescence may be used to measure the antigen binding activity of the antibody of the invention. In ELISA, the antibody of the present invention is immobilized on a plate, a peptide of the invention is applied to the plate, and then a sample containing a desired antibody, such as culture supernatant of antibody producing cells or purified antibodies, is applied. Then, a secondary antibody that recognizes the primary antibody and is labeled with an enzyme, such as alkaline phosphatase, is applied, and the plate is incubated. Next, after washing, an enzyme substrate, such as p-nitrophenyl phosphate, is added to the plate, and the absorbance is measured to evaluate the antigen binding activity of the sample. A fragment of the peptide, such as a C-terminal or N-terminal fragment, may be used as the antigen to evaluate the binding activity of the antibody. BIAcore (Pharmacia) may be used to evaluate the activity of the antibody according to the present invention.

The above methods allow for the detection or measurement of a peptide of the invention, by exposing an antibody of the invention to a sample presumed to contain a peptide of the invention, and detecting or measuring the immune complex formed by the antibody and the peptide.

Because the method of detection or measurement of the peptide according to the invention can specifically detect or measure a peptide, the method may be useful in a variety of experiments in which the peptide is used.

### XII. VECTORS AND HOST CELLS

The present invention also provides a vector and host cell into which a nucleotide encoding the peptide of the present invention is introduced. A vector of the present invention is useful to keep a nucleotide, especially a DNA, of the present invention in host cell, to express the peptide of the present invention, or to administer the nucleotide of the present invention for gene therapy.

When E. coli is a host cell and the vector is amplified and produced in a large amount in E. coli (e.g., JM109, DH5 alpha, HB101 or XL1Blue), the vector should have “ori” to be amplified in E. coli and a marker gene for selecting transformed E. coli (e.g., a drug-resistance gene selected by a drug such as ampicillin, tetracycline, kanamycin, chloramphenicol or the like). For example, M13-series vectors, pUC-series vectors, pBR322, pBluescript, pCR-Script, etc. can be used. In addition, pGEM-T, pDIRECT and pT7 can also be used for subcloning and extracting cDNA as well as the vectors described above. When a vector is used to produce the protein of the present invention, an expression vector is especially useful.

For example, an expression vector to be expressed in E. coli should have the above characteristics to be amplified in E. coli. When E. coli, such as JM109, DH5 alpha, HB101 or XL1 Blue, are used as a host cell, the vector should have a promoter, for example, lacZ promoter (Ward et al., Nature 341: 544-6 (1989); FASEB J 6: 2422-7 (1992)), araB promoter (Better et al., Science 240: 1041-3 (1988)), T7 promoter or the like, that can efficiently express the desired gene in E. coli. In that respect, pGEX-5X-1 (Pharmacia), “QIAexpress system” (Qiagen), pEGFP and pET (in this case, the host is preferably BL21 which expresses T7 RNA polymerase), for example, can be used instead of the above vectors. Additionally, the vector may also contain a signal sequence for peptide secretion. An exemplary signal sequence that directs the peptide to be secreted to the periplasm of the E. coli is the pelB signal sequence (Lei et al., J Bacteriol 169: 4379 (1987)). Means for introducing of the vectors into the target host cells include, for example, the calcium chloride method, and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vector derived from yeast (e.g., “Pichia Expression Kit” (Invitrogen), pNV11, SP-Q01) and expression vectors derived from Bacillus subtilis (e.g., pPL608, pKTH50) can be used for producing the polypeptide of the present invention.

In order to express the vector in animal cells, such as CHO, COS or NIH3T3 cells, the vector should have a promoter necessary for expression in such cells, for example, the SV40 promoter (Mulligan et al., Nature 277: 108 (1979)), the MMLV-LTR promoter, the EF1 alpha promoter (Mizushima et al., Nucleic Acids Res 18: 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13.

The following examples are presented to illustrate the present invention and to assist one of ordinary skill in making and using the same. The examples are not intended in any way to otherwise limit the scope of the invention.

### EXAMPLES

### Materials and Methods

Cell Lines

T2 (HLA-A2), human B-lymphoblastoid cell line, and COST, African green monkey kidney cell line, were purchased from ATCC.

Candidate Selection of Peptides Derived from MYBL2

9-mer and 10-mer peptides derived from MYBL2 that bind to HLA-A*0201 molecule were predicted using “NetMHC 3.0” binding prediction server (http://www.cbs.dtu.dk/services/NetMHC/) (Buus et al. (Tissue Antigens, 62:378-84, 2003), Nielsen et al. (Protein Sci., 12:1007-17, 2003, Bioinformatics, 20(9):1388-97, 2004)). These peptides were synthesized by Biosynthesis (Lewisville, Tex.) according to a standard solid phase synthesis method and purified by reversed phase high performance liquid chromatography (HPLC). The purity (>90%) and the identity of the peptides were determined by analytical HPLC and mass spectrometry analysis, respectively. Peptides were dissolved in dimethylsulfoxide (DMSO) at 20 mg/ml and stored at −80 degrees C.

In Vitro CTL Induction

Monocyte-derived dendritic cells (DCs) were used as antigen-presenting cells (APCs) to induce cytotoxic T lymphocyte (CTL) responses against peptides presented on human leukocyte antigen (HLA). DCs were generated in vitro as described elsewhere (Nakahara S et al., Cancer Res 2003 Jul. 15, 63(14): 4112-8). Specifically, peripheral blood mononuclear cells (PBMCs) isolated from a normal volunteer (HLA-A*0201 positive) by Ficoll-Plaque (Pharmacia) solution were separated by adherence to a plastic tissue culture dish (Becton Dickinson) so as to enrich them as the monocyte fraction. The monocyte-enriched population was cultured in the presence of 1000 U/ml of granulocyte-macrophage colony-stimulating factor (R&D System) and 1000 U/ml of interleukin (IL)-4 (R&D System) in AIM-V Medium (Invitrogen) containing 2% heat-inactivated autologous serum (AS). After 7 days of culture, the cytokine-induced DCs were pulsed with 20 microgram/ml of each of the synthesized peptides in the presence of 3 microgram/ml of beta 2-microglobulin for 3 hrs at 37 degrees C. in AIM-V Medium. The generated cells appeared to express DC-associated molecules, such as CD80, CD83, CD86 and HLA class II, on their cell surfaces (data not shown). These peptide-pulsed DCs were then inactivated by X-irradiated (20 Gy) and mixed at a 1:20 ratio with autologous CD8+ T cells, obtained by positive selection with CD8 Positive Isolation Kit (Dynal). These cultures were set up in 48-well plates (Corning); each well contained 1.5×10⁴ peptide-pulsed DCs, 3×10⁵ CD8+ T cells and 10 ng/ml of IL-7 (R&D System) in 0.5 ml of AIM-V/2% AS medium. Three days later, these cultures were supplemented with IL-2 (CHIRON) to a final concentration of 20 IU/ml. On days 7 and 14, the T cells were further stimulated with the autologous peptide-pulsed DCs. The DCs were prepared each time by the same way described above. CTL was tested against peptide-pulsed T2 cells after the 3rd round of peptide stimulation on day 21 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

CTL Expansion Procedure

CTLs were expanded in culture using the method similar to the one described by Riddell et al. (Walter E A et al., N Engl J Med 1995 Oct. 19, 333(16): 1038-44; Riddell S R et al., Nat Med 1996 February, 2(2): 216-23). A total of 5×10⁴ CTLs were suspended in 25 ml of AIM-V/5% AS medium with 2 kinds of human B-lymphoblastoid cell lines, inactivated by Mitomycin C, in the presence of 40 ng/ml of anti-CD3 monoclonal antibody (Pharmingen). One day after initiating the cultures, 120 IU/ml of IL-2 were added to the cultures. The cultures were fed with fresh AIM-V/5% AS medium containing 30 IU/ml of IL-2 on days 5, 8 and 11 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 microliter/well of AIM-V Medium containing 5% AS. 50 microliter/well of IL-2 were added to the medium 10 days later so to reach a final concentration of 125 U/ml IL-2. CTL activity was tested on the 14th day, and CTL clones were expanded using the same method as described above (Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Specific CTL Activity

To examine specific CTL activity, interferon (IFN)-gamma enzyme-linked immunospot (ELISPOT) assay and IFN-gamma enzyme-linked immunosorbent assay (ELISA) were performed. Specifically, peptide-pulsed T2 (1×10⁴/well) was prepared as stimulator cells. Cultured cells in 48 wells were used as responder cells. IFN-gamma ELISPOT assay and IFN-gamma ELISA assay were performed under manufacture procedure.

Establishment of the Cells Forcibly Expressing Either or Both of the Target Gene and HLA-A02

The cDNA encoding an open reading frame of target genes or HLA-A*0201 was amplified by PCR. The PCR-amplified product was cloned into expression vector. The plasmids were transfected into COS7, which is the target genes and HLA-A*0201-null cell line, using lipofectamine 2000 (Invitrogen) according to the manufacturer's recommended procedures. After 2 days from transfection, the transfected cells were harvested with versene (Invitrogen) and used as the stimulator cells (5×10⁴ cells/well) for CTL activity assay.

Results

Prediction of HLA-A02 Binding Peptides Derived from MYBL2

Tables 1a and 1b show the HLA-A02 binding 9mer and 10mer peptides of MYBL2 in the order of high binding affinity. A total of 4 peptides with potential HLA-A02 binding ability were selected and examined to determine the epitope peptides.

TABLE 1a
HLA-A02 binding 9mer peptides derived from MYBL2

| Start | Amino acid | | SEQ |
|---|---|---|---|
| Position | sequence | Kd (nM) | ID NO |
| 355 | VLPPRQPSA | 213 | 1 |
| 597 | LMMSTLPKS | 285 | 2 |
| 144 | RIICEAHKV | 493 | 3 |

TABLE 1b
HLA-A02 binding 10mer peptides derived from MYBL2

| Start Position | Amino acid sequence | Kd (nM) | SEQ ID NO |
|---|---|---|---|
| 596 | KLMMSTLPKS | 226 | 4 |

Start position indicates the number of amino acid residue from the N-terminus of MYBL2.

Binding dissociation constant [Kd (nM)] is derived from “NetMHC3.0”.

CTL Induction with the Predicted Peptides from MYBL2 Restricted with HLA-A*0201

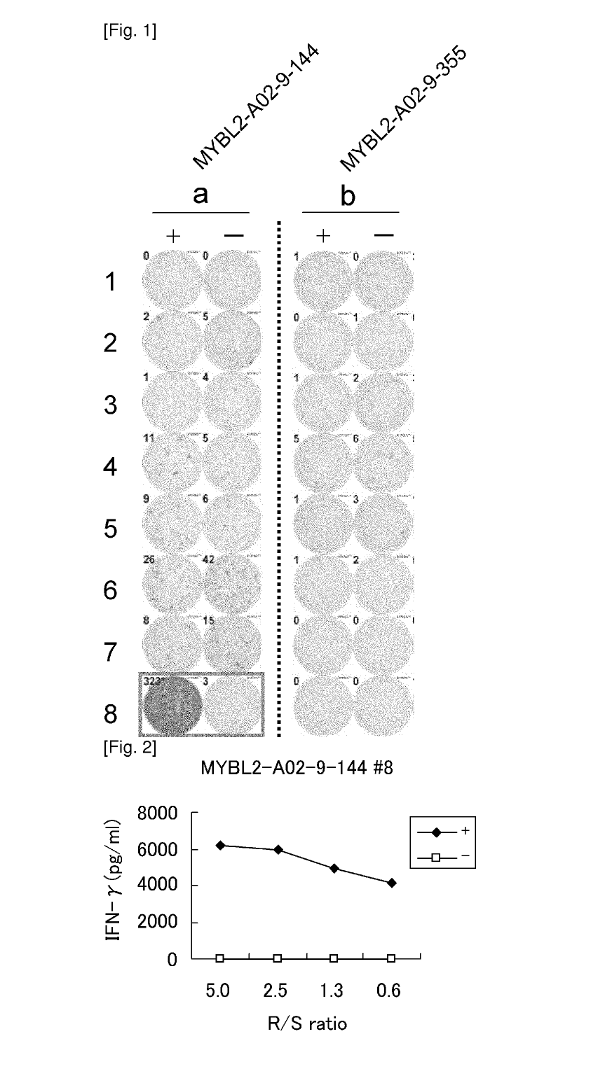

CTLs for those peptides derived from MYBL2 were generated according to the protocols as described in “Materials and Methods”. Peptide specific CTL activity was determined by IFN-gamma ELISPOT assay (FIG. 1). Well number #8 stimulated with MYBL2-A02-9-144 (SEQ ID NO: 3) (a) demonstrated potent IFN-gamma production as compared to the control wells. On the other hand, no specific CTL activity was determined by stimulation with other peptides shown in Table 1, despite those peptides had possible binding activity with HLA-A*0201. As a typical case of negative data, specific IFN-gamma production was not observed from the CTL stimulated with MYBL2-A02-9-355 (SEQ ID NO: 1) (b). As a result, it indicated that MYBL2-A02-9-144 (SEQ ID NO: 3) derived from MYBL2 was screened as the peptides that could induce potent CTLs.

Establishment of CTL Line and Clone Against MYBL2 Specific Peptide

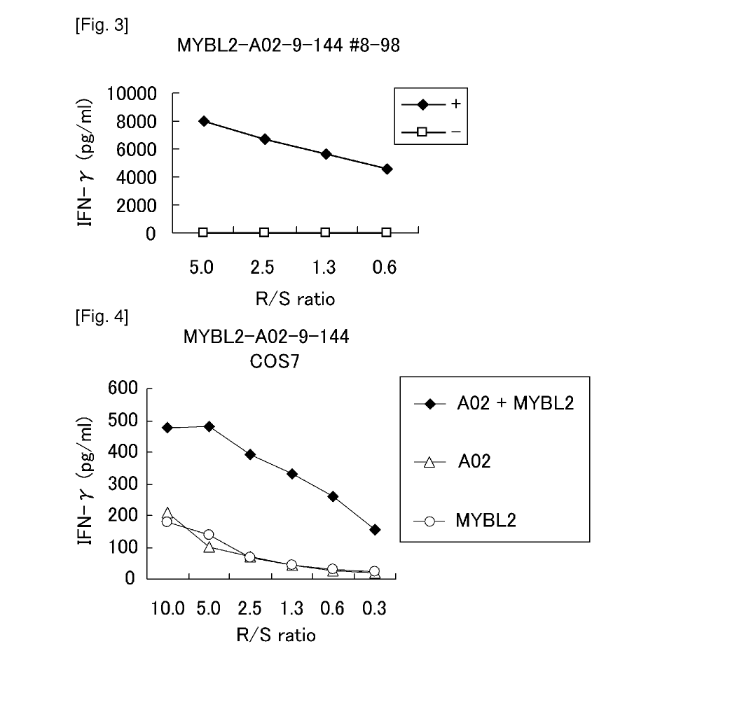

The cells that showed peptide specific CTL activity detected by IFN-gamma ELISPOT assay in the well number #8 with MYBL2-A02-9-144 (SEQ ID NO: 3) were expanded and the CTL line was established by expansion procedure as described in the “Materials and Methods” section above. CTL activity of this CTL line was determined by IFN-gamma ELISA assay (FIG. 2). The CTL line demonstrated potent IFN-gamma production against the target cells pulsed with the corresponding peptide as compared to target cells without peptide pulse. Furthermore, the CTL clone was established by limiting dilution from the CTL line as described in “Materials and Methods”, and IFN-gamma production from the CTL clone against target cells pulsed peptide was determined by IFN-gamma ELISA assay. Potent IFN-gamma productions were determined from the CTL clone stimulated with MYBL2-A02-9-144 (SEQ ID NO: 3) (FIG. 3).

Specific CTL Activity Against Target Cells Exogenously Expressing MYBL2 and HLA-A*0201

The established CTL clone raised against MYBL2-A02-9-144 (SEQ ID NO: 3) was examined for the ability to recognize target cells that express MYBL2 and HLA-A*0201 molecule. Specific CTL activity against COS7 cells which transfected with both the full length of MYBL2 and HLA-A*0201 molecule gene (a specific model for the target cells that express MYBL2 and HLA-A*0201 gene) was tested by using the CTL clone raised by MYBL2-A02-9-144 (SEQ ID NO: 3) as the responder cells. COS7 cells transfected with either full length of MYBL2 or HLA-A* 0201 were prepared as the controls. In FIG. 4, the CTL clone stimulated with MYBL2-A02-9-144 (SEQ ID NO: 3) showed potent CTL activity against COS7 cells expressing both MYBL2 and HLA-A* 0201. On the other hand, no significant specific CTL activity was detected against the controls. Thus, these data clearly demonstrated that MYBL2-A02-9-144 (SEQ ID NO: 3) was naturally expressed on the target cells with HLA-A*0201 molecule and were recognized by the CTLs. These results indicate that MYBL2-A02-9-144 (SEQ ID NO: 3) derived from MYBL2 may be suitable as a cancer vaccine for the treatment of patients with MYBL2 expressing tumors.

Homology Analysis of Antigen Peptides

The CTLs stimulated with MYBL2-A02-9-144 (SEQ ID NO: 3) showed significant and specific CTL activity. This result may be due to the fact that the sequence of MYBL2-A02-9-144 (SEQ ID NO: 3) is homologous to peptide derived from other molecules that are known to sensitize the human immune system. To exclude this possibility, homology analyses were performed for this peptide sequence using as queries the BLAST algorithm (http://www.ncbi.nlm.nih.gov/blast/blast.cgi) which revealed no sequence with significant homology. The results of homology analyses indicate that the sequence of MYBL2-A02-9-144 (SEQ ID NO: 3) is unique and thus, there is little possibility, to our best knowledge, that this molecules raise unintended immunologic response to some unrelated molecule.

In conclusion, MYBL2-A02-9-144 (SEQ ID NO: 3) was identified as a novel HLA-A*0201 epitope peptide derived from MYBL2. The results herein demonstrate that MYBL2-A02-9-144 (SEQ ID NO: 3) may be suitable for use in cancer immunotherapy.

### INDUSTRIAL APPLICABILITY

The present invention describes new TAAs, particularly those derived from MYBL2 that induce potent and specific anti-tumor immune responses and have applicability to a wide array of cancer types. Such TAAs warrant further development as peptide vaccines against diseases associated with MYBL2, e.g., cancer, more particularly, testicular tumor, pancreatic cancer, bladder cancer, non-small cell lung cancer, small cell lung cancer and esophageal cancer.

While the invention is herein described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention, the metes and bounds of which are defined by the appended claims.

1. An isolated oligopeptide of (a) or (b) below:
(a) an isolated oligopeptide comprising an amino acid sequence selected from the group consisting of SEQ ID NOs: 2, 3 and 4;
(b) an isolated oligopeptide consisting of an amino acid sequence selected from the group consisting of SEQ ID NOs: 2, 3 and 4, in which 1, 2, or several amino acids are inserted, substituted, deleted or added;
wherein said oligopeptide binds an HLA antigen and has cytotoxic T lymphocyte (CTL) inducibility.

2. (canceled)

3. The oligopeptide of claim 1, wherein said oligopeptide has one or both of the following characteristics:
(a) the second amino acid from the N-terminus is selected from among leucine and methionine; and
(b) the C-terminal amino acid is selected from among valine and leucine.

4. The isolated oligopeptide of claim 1, wherein the HLA antigen is HLA-A2.

5. The isolated oligopeptide of claim 1, wherein said oligopeptide is a nonapeptide or decapeptide.

6. An isolated polynucleotide encoding an oligopeptide of claim 1.

7. An agent for inducing CTL, wherein the agent comprises one or more of the oligopeptide(s) set forth in claim 1, or one or more of the polynucleotides encoding said peptide(s).

8. A pharmaceutical agent for the treatment and/or prophylaxis of cancer, and/or the prevention of a postoperative recurrence thereof, wherein the agent comprises one or more of the oligopeptide(s) set forth in claim 1, or one or more of the polynucleotides encoding said peptide(s).

9. The pharmaceutical agent of claim 8, which is formulated for the administration to a subject whose HLA antigen is HLA-A2.

10. The pharmaceutical agent of claim 8, which is formulated for treating cancer.

11. A method for inducing an antigen-presenting cell (APC) with CTL inducibility, wherein the method comprises one of the following steps:
(a) contacting an APC with an oligopeptide of claim 1 in vitro, ex vivo or in vivo; or
(b) introducing a polynucleotide encoding an oligopeptide of claim 1 into an APC.

12. A method for inducing CTL by any of the methods comprising at least one of the following steps:
(a) co-culturing CD8-positive T cells with APCs, which presents on its surface a complex of an HLA antigen and an oligopeptide of claim 1;
(b) co-culturing CD8-positive T cells with exosomes, which presents on its surface a complex of an HLA antigen and an oligopeptide of claim 1; and
(c) introducing a gene that comprises a polynucleotide encoding a T cell receptor (TCR) subunit polypeptide binding to an oligopeptide of claim 1 into a T cell.

13. An isolated APC that presents on its surface a complex of an HLA antigen and an oligopeptide of claim 1.

14. The APC of claim 13, which is induced by a method for inducing an antigen-presenting cell (APC) with CTL inducibility, wherein the method comprises one of the following steps:
(a) contacting an APC with said oligopeptide in vitro, ex vivo or in vivo; or
(b) introducing a polynucleotide encoding said oligopeptide into an APC.

15. An isolated CTL that targets a peptide of claim 1.

16. The CTL of claims 15, which is induced by a method for inducing CTL by any of the methods comprising at least one of the following steps:
(a) co-culturing CD8-positive T cells with APCs, which presents on their surface a complex of an HLA antigen and said oligopeptide;
(b) co-culturing CD8-positive T cells with exosomes, which presents on their surface a complex of an HLA antigen and said oligopeptide; and
(c) introducing a gene that comprises a polynucleotide encoding a T cell receptor (TCR) subunit polypeptide binding to said oligopeptide into a T cell.

17. A method of inducing immune response against cancer in a subject comprising the step of: administering to the subject an agent comprising an oligopeptide of claim 1, an immunologically active fragment thereof, or a polynucleotide encoding the oligopeptide or immunologically active fragment.

18. The pharmaceutical agent of claim 9, which is formulated for treating cancer.
